(19) United States
(12) Patent Application Publication
(10) Pub. No.: US 2005/0079520 A1
(43) Pub. Date: Apr. 14, 2005

(54) Multiplexed analyte detection

(76) Inventor: Jie Wu, Foster City, CA (US)

Correspondence Address: MORRISON & FOERSTER LLP, 755 PAGE MILL RD, PALO ALTO, CA, 94304-1018, US

(21) Appl. No.: 10/897,191

(22) Filed: Jul. 21, 2004

Related U.S. Application Data
(60) Provisional application No. 60/489,213, filed on Jul. 21, 2003.

(51) Int. Cl. C12Q 1/68; C12P 19/34

(52) U.S. Cl. 435/6; 435/91.2

(57) ABSTRACT

The invention provides methods and kits for quantitating or detecting the presence of a plurality of different target molecules in a sample using detector molecules comprising nucleic acid tags.

### CROSS-REFERENCE TO RELATED APPLICATIONS

This application claims the benefit of U.S. provisional application Ser. No. 60/489,213, filed Jul. 21, 2003, which is incorporated in its entirety by reference.

### FIELD OF THE INVENTION

The invention relates to a method for detecting the presence of multiple target molecules in a sample which may contain the target molecules, using nucleic acid-containing detector molecules, amplification and quantitation or detection of the detector molecules.

### BACKGROUND OF THE INVENTION

Numerous assays are known for detecting analytes in a sample. Two general types of analytes are protein analytes and nucleic acid analytes. The technology and assays directed at detecting proteins have historically developed separately and largely independently from the technology and assays directed at detecting nucleic acids. Several reasons exist for this trend in these two fields. Initially, proteins and nucleic acids are chemically distinct and have very different chemical and physical properties. Assays to detect proteins were developed first, due in part to the presence and stability of proteins in the blood, urine, saliva, etc., samples which are readily available, and to the early correlation of physiological condition or disease with the presence proteins. Nucleic acids are generally less stable under assay conditions and are not found readily in free form in body fluids. Assays to detect nucleic acid analytes were developed much later and substantially independently from the protein assays. Assays for nucleic acids have been cumbersome, with low through-put, poor specificity and poor quantitative ability. Currently, protein analytical assays and nucleic acid analytical assays are considered to be two separate fields and practitioners in the two fields do not look to the literature of the other field for guidance in solving problems.

Early protein assays relied on the ability of antibodies to bind to specific protein analytes with sufficiently low dissociation constant (Kd) and with adequate specificity. Antibody capture assays are an easy and convenient screening method. In an antibody capture assay, an antigen is bound to a solid substrate, detection antibodies are allowed to bind to the antigen and then unbound antibodies are removed by washing. The bound antibodies are then detected using a detector molecule which specifically recognizes the antibody. Most antibody capture assays rely on an indirect method of detecting the antibody. For example, where the antibody is a murine antibody, the detector molecule might be a rabbit anti-mouse antibody which has been labeled with a detectable tag. Conventionally detectable tags have included radioactive isotopes, dyes and enzymes which act on a substrate to produce a detectable molecule, e.g., a chromogen.

One disadvantage of the antibody capture assay as described above is that the target molecules in the sample must be immobilized onto a solid support. Alternatively, in an antigen capture assay, the detection method identifies the presence of an antigen in a sample without the necessity of immobilizing the antigen and other molecules onto the support. In these methods, an antibody called a “capture antibody” is bound to a solid support initially and then the antigen is allowed to react with another antibody, called the detection antibody, to form a complex and the complex is subsequently detected. This is also known as a “sandwich assay” since the two antibodies form a sandwich around the antigen (Burgess, 1988.). The two antibodies in a sandwich assay must react with different regions (epitopes) of the target protein. For multimeric target proteins, the capture antibody and the detection antibody may have the same specificity, since there is more than one copy of the target epitope per oligomeric target molecule. However, for monomeric target molecules such as monomeric proteins, the capture antibody and the detection antibody must recognize different, non-overlapping epitopes. Alternatively, an antibody-antigen complex may be formed prior to binding of the antibody to a solid phase followed by detection of the complex.

Although assays using antibodies are very useful, it is generally accepted that the detection limit of an assay is limited by the Kd of the antibody used as the capture molecule (Griswold, W. (1987) J. Immunoassay 8:145-171; O'Connor, T., et al. (1995) Biochem. Soc. Trans. 23(2): 393S). In practice, the detection limit of these assays is approximately 1% of the capture antibody Kd. As the concentration of analyte decreases to this sensitivity limit, the low percentage of capture molecules with bound analyte is insufficient to produce a detectable signal to noise ratio. For this reason, antibody-based assays using state of the art fluorimetric or chemiluminescent detection systems have a detection limit of about 1 pg/ml (10 e-14 M for an “average” protein of molecular weight 50,000 daltons). See also Tijssen, P., Practice and Theory of Enzyme Immunoassays in Laboratory Techniques in Biochemistry and Molecular Biology, vol. 15, ed. by Burdon, R. H. and van Knippenberg, P. H., Elsevier, N.Y., 1985, pp. 132-136.

The detection of nucleic acid analytes requires different technology. In the mid-1980's, research in DNA technology lead to a method in which DNA could be amplified through a repeated enzymatic amplification process (Saiki et al., 1985; Mullis et al., 1987). The process, later named the polymerase chain reaction (PCR), uses two complementary oligonucleotide sequences (called primers) that flank the region of interest (5′ to 3′). The enzymatic process began with a denaturation step in the presence of the primers, then the temperature was lowered to allow primer annealing and Klenow fragment DNA polymerase I was added to extend the primers. Through repeated denaturation, annealing and extension of the desired fragment, exponential amplification of the target DNA was achieved. Many improvements have been made to PCR, but one important change was the incorporation of a DNA polymerase from Thermus aquaticus (Taq), a thermophilic bacterium (Saiki et al., 1988). Taq polymerase is a thermostable polymerase and is nearly unaffected by the denaturation steps involved in PCR, an improvement over the previous system where Klenow DNA polymerase I would have to be added to the reaction periodically because the enzyme did not tolerate the denaturation step and lost activity.

In this type of enzymatic reaction, under conditions that allow primers to anneal efficiently, an exponential accumulation or a doubling of the template occurs every cycle. Such amplification allows for extremely low detection levels, some claiming to be able to detect single amplicons in a background of many other DNA molecules (Lentz et al., 1997).

Conventional PCR amplification is not a quantitative detection method, however. During amplification, primer dimers and other extraneous nucleic acids are amplified together with the nucleic acid corresponding to the analyte. These impurities must be separated, usually with gel separation techniques, from the amplified product resulting in possible losses of material. Although methods are known in which the PCR product is measured in the log phase (Kellogg et al., 1990; Pang et al, 1990), these methods require that each sample have equal input amounts of nucleic acid and that each sample amplifies with identical efficiency, and are therefore, not suitable for routine sample analyses. To allow an amount of PCR product to form which is sufficient for later analysis and to avoid the difficulties noted above, quantitative competitive PCR amplification uses an internal control competitor and is stopped only after the log phase of product formation has been completed (Becker-Andre, 1991; Piatalc et al., 1993a,b).

In one application of PCR as an amplification system (Sano et al., 1992), an immuno-PCR method was developed that linked a microplate assay for a specific analyte with the amplification power of PCR for detection. The method detected, but did not quantitate, Bovine Serum Albumin (BSA) passively absorbed to an immuno-assay plate. Using an antibody specific for BSA, then bridging a biotin-labeled reporter amplicon with a protein A-streptavidin fusion protein, the assay utilized PCR amplification to detect several hundred molecules of BSA by agarose gel analysis of the reporter amplicon. However, this method could not be applied to biological samples due to the absence of a specific analyte capture molecule. Others have improved the method by substituting the protein A-streptavidin fusion protein, which was not widely available, with a biotinylated secondary antibody and a streptavidin bridge to bind the biotinylated reporter amplicon (Zhou et al., 1993). The addition of the five assay reagents plus washing, PCR amplification and detection resulted in an assay that was laborious and was subjected to both stoichiometric and disassociation complications (Hendrickson et al., 1995). Another improvement in this assay approach came with the development of a method to covalently link a reporter amplicon to the secondary antibody (Hendrickson et al., 1995; Hnatowich et al., 1996). The direct linkage of the amplicon decreased the reagents used in the assay and the stoichiometric and disassociation complications that can occur. However, these methods still require significant post-PCR manipulations, adding to increased labor and the very real possibility of laboratory contamination.

Sandwich immuno-PCR is a modification of the conventional ELISA format in which the detecting antibody is labeled with a DNA label, and is applicable to the analysis of biological samples. In an early format of an antibody sandwich immuno-PCR, primary antibody was immobilized to a plate and sequentially, the sample, biotinylated detecting antibody, streptavidin, and biotinylated DNA, were added. This format was later improved by the direct conjugation of the DNA to the antibody and replacement of the gel electrophoresis by using labeled primers to generate a PCR product that can be assayed by ELISA (Niemeyer et al., 1996). The amplification ability of PCR provides large amounts of the DNA label which can be detected by various methods, typically gel electrophoresis with conventional staining (T. Sano et al., 1992, Science, 258:120-122). Replication of the antibody-borne DNA label using PCR provides enhanced sensitivity for antigen detection. Immuno-PCR techniques have been extended to the detection of multiple analytes (Joerger et al., 1995; Hendrickson, 1995). While immuno-PCR has provided sensitivities exceeding those of conventional ELISA, purification of the amplified product by gel electrophoresis requires substantial human manipulation and is, therefore, time-consuming. Further, the primers used in the PCR amplification step may dimerize and the dimers are amplified under the PCR conditions leading to side products which compete for PCR amplification. In addition, matrix nucleic acids and other contaminating nucleic acids may be present or introduced and will be amplified by PCR.

Using a primary capture antibody, immuno-PCR methods and reagents are similar to a direct sandwich antigen ELISA, the difference coming at the choice of the detection method. Immuno-PCR methods have been successful and claim to obtain attomole level of sensitivity in some cases, including the detection of the following analytes: tumor necrosis factor (Sanna et al., 1995), beta-galactosidase (Hendrickson et al., 1995), human chorionic gonadotropin, human thyroid stimulating hormone, soluble murine T-cell receptor (Sperl et al., 1995), recombinant hepatitis B surface antigen (Miemeyer et al., 1995), human atrial natriuretic peptide (Numata et al., 1997) and beta-glucuronidase (Chang et al., 1997).

In immuno-PCR, antigen concentrations are generally determined by post PCR analysis of the reporter amplicon by either gel electrophoresis or PCR-ELISA. Quantitation of the DNA label by analyzing the endpoint PCR product is prone to errors since the rate of product formation decreases after several cycles of logarithmic growth (Ferre, 1992; Raeymakers et al., 1995) and the post PCR sample handling may lead to laboratory contamination. In addition, these methods require multiple steps and washes, during which the antibody:antigen complex may dissociate (Tijssen, P., ibid.).

Another method for amplicon quantitation, e.g. quantitative competitive PCR, uses laser induced capillary electrophoresis techniques to assess fluorescent PCR products (Fasco et al., 1995; Williams et al., 1996). In the context of a immuno-PCR analysis, all of these amplicon quantitation techniques require significant post PCR analysis and induce the possibility of PCR product contamination of the laboratory for following assays because of the handling requirements. Furthermore, these techniques are only able to analyze end-point PCR, PCR that has been stopped at a fixed PCR cycle number (e.g. 25 cycles of PCR). This poses a problem in the dynamic range of amplicon quantitation because only some PCR reactions may be in the log phase of amplification; reactions with high amounts of template will have used all PCR reagents and stopped accumulating amplicon exponentially and reactions with low amounts of template might not have accumulated enough amplicon to be detectable. Therefore, this phenomenon limits the detection range of PCR and can limit the analyte detection range of immuno-PCR assays.

In a further development of PCR technology, real time quantitative PCR has been applied to nucleic acid analytes (Heid et al., 1996). In this method, PCR is used to amplify DNA in a sample in the presence of a nonextendable dual labeled fluorogenic hybridization probe. One fluorescent dye serves as a reporter and its emission spectra is quenched by the second fluorescent dye. The method uses the 5′ nuclease activity of Taq polymerase to cleave a hybridization probe during the extension phase of PCR. The nuclease degradation of the hybridization probe releases the quenching of the reporter dye resulting in an increase in peak emission from the reporter. The reactions are monitored in real time. Reverse transcriptase (RT)-real time PCR (RT-PCR) has also been described (Gibson et al., 1996). The Sequence Detection system (ABI Prism, ABD of Perkin Elmer, Foster City, Calif.) uses a 96-well or 384-well thermal cycler that can monitor fluorescent spectra in each well continuously in the PCR reaction, therefore the accumulation of PCR product can be monitored in ‘real time’ without the risk of amplicon contamination in the laboratory.

The Sequence Detection system takes advantage of a fluorescence energy theory known as Forster-type energy transfer (Lakowicz et al., 1983). The PCR reaction contains a fluorescently dual-labeled non-extendible probe that binds to the specific target between the PCR primers. The probe commonly contains a FAM (6-carboxyfluorescein) on the 5′-end and a TAMRA (6-carboxy-tetramethylrh-odamine) on the 3′-end. When the probe is intact, the FAM dye (reporter dye) fluorescence emission is quenched by the proximity of the TAMRA dye (quencher dye) through Forster-type energy transfer. As PCR cycling continues, amplicon is produced and the hybridized probe is cleaved by the use of a polymerase that contains the 5′-3′ nuclease activity which chews through the probe, hence the nickname ‘TaqMan.RTM.’ given to the machine. With the cleavage of the probe, the reporter dye is then physically separated from the quencher dye, resulting in an increase in FAM fluorescence because of decreased quenching by TAMRA. The system uses an argon ion laser for fluorescence excitation (488 nm) and a charge-coupled device (CCD) camera to monitor the PCR reactions and collect fluorescence emission over the range of 500 nm to 660 nm for all 96 or 384 wells (SDS User's Manual). Using an algorithm that takes into account the overlapping emission spectra of the dyes used on the machine, the raw fluorescence data can be determined for the reporter, quencher and passive internal reference (ROX, 6-carboxy-X-rhodamine) dyes. The reference dye is used to normalize cycle to cycle fluorescence variations in each well. The Sequence Detection application then calculates a normalized change in reporter fluorescence (ARn) as follows:

ΔRₙ=ΔRₙ⁺−ΔRₙ⁻

where the ΔRₙ⁺is the ‘reporter's emission fluorescence’/‘passive internal reference fluorescence’ for that particular PCR cycle and ΔRₙ⁻ is the ‘reporter's emission fluorescence’/passive internal reference ‘fluorescence’ for a predetermined background period of the PCR reaction (typically cycles 3-15). Plotting the ΔRₙ versus PCR cycle reveals an amplification plot that represents the accumulation of the amplicon in the PCR reaction and cleavage of the probe. Using the provided software, the threshold value is either set manually by the user (at a fixed ΔRₙ value) or calculated, typically at 10 standard deviations above the mean of the background period of PCR (ΔRₙ⁻). The point on the amplification plot at which a sample's fluorescence intersects the threshold value is referred to as the Cₜ value (PCR Cycle threshold) for that sample. Relative amounts of PCR product are compared among PCR reactions using the calculated Cₜ value. Using the Sequence Detection system, DNA and RNA have been successfully used for quantitative PCR (Heid et al., 1996) and rt-PCR (Gibson et al., 1996).

Nucleic acids have also been used as detector molecules in assays. The idea of “in vitro genetics” has been used to describe the isolation of binding nucleic acid ligands (Szostak et al., 1992). In general, the method involves taking a pool of very diverse nucleic acid sequences (typically degenerate oligonucleotides), introducing these sequences to a target and separating the bound sequences from the unbound sequences. The separation of the bound sequences results in a new pool of oligonucleotides that have been maturated by their preference to interact with the target, a type of genetic selection performed on the lab bench.

Nucleic acid and protein interactions in the cell are not uncommon occurrences. It is known that nucleic acids can fold to form secondary and tertiary structures and that these structures are important for binding interactions with proteins (Wyatt et al., 1989). The maturation of nucleic acid-protein binding interactions has been examined in vitro by varying the sequence of nucleic acid ligands (Tuerk et al., 1990). A technique known as SELEX (Systematic Evolution of Ligands by EXponential enrichment) is used to isolate novel nucleic acid ligands to a target of choice. These ligands were referred to as aptamers. The Greek root ‘apta’, meaning “to fit”, suggests a method for which the nucleic acid may fold and fit into pockets on target molecules. See U.S. Pat. No. 5,652,107; U.S. Pat. No. 5,631,146; U.S. Pat. No. 5,688,670; U.S. Pat. No. 5,652,107; A. D. (Ellington et al., 1992; Ellington et al., 1990; Greene et al., 1991).

The power of maturing nucleic acid ligand pools in the SELEX procedure involves separating ligand-target complexes from free nucleic acid sequences. In the selection described above, a membrane that has a higher affinity for protein than RNA was used to create a new matured pool biased for sequences that interact with the protein. Maturation of the selection pool is accelerated by creating competition among the diverse RNA ligands in the pool. By lowering the target concentration, a situation is created where the binding sites are limited. The competition for these binding sites promotes higher affinity ligand selection. An unfortunate problem in some selections is the maturation of non-specific ligands, or ligands that bind to the nitrocellulose filter or other material in the selection procedure. One method used to avoid such ligands involves the use of carrier nucleic acid that cannot be extended by the selection PCR primer set (such as tRNA). Other methods of selection involve alternative procedures to separate ligand-target complexes, such as; affinity column binding, gel-shift assays and immuno-assay capture. The design of a nucleic acid library involves three main considerations; minimizing amplification artifacts (resulting from miss priming), amount of randomness and length of the random region (Conrad et al., 1996). In designing a PCR amplification system, primer design is important to optimize the amplification of the specific amplicon of choice and to minimize non-specific amplification of other products by miss priming (either to amplicons of non-interest or primer-primer annealing). Primer design is also important in aptamer library design because of the large number of PCR cycles that are performed. A typical SELEX round will include 12-25 PCR cycles and a SELEX selection might include as much as 15 rounds, resulting in over 200 cycles of PCR on the selection pool. It is clear that miss primed PCR artifacts will accumulate in the selection pool that is subjected to such a large amount of PCR cycling. The amount of randomness can play an important role in the selection library if the study protein has a known nucleic acid sequence (such as the T-DNA polymerase selection above). These libraries can be completely randomized in certain regions (keeping other wild-type sequence intact for secondary structure), or the wild-type sequences can be “doped” to contain a higher percentage of natural bases and a lower percentage of random bases (e.g. 70% G's and 10% A, C or T). Finally, the length of the random region can be varied over a wide range when using proteins that have known nucleic acid interactions. When selecting aptamers with proteins that have no known natural nucleic acid ligands, completely randomized libraries can be used, although the length must be considered. In longer completely randomized pools, greater secondary structure can be obtained because more bases are available. In shorter randomized pools, simpler secondary structure is obtained but a greater representation of all sequence possibilities is achieved (because of the physical limitation in the amount of DNA/RNA that one can select for in the first round; Ellington et al, 1994).

Since the original SELEX experiments, many nucleic acid ligands have been selected to a large variety of targets. Many proteins that normally bind nucleic acids have been shown to be good candidates for these SELEX selections. The designs of such selections have ranged from randomizing only the known binding region (T4 DNA Polymerase) to selections on completely randomigzed libraries. Other examples include; bacteriophage R17 coat protein (Schneider et al., 1992), E. Coli rho factor (Schneider et al., 1993), E. Coli ribosomal protein S1 (Ringquist et al., 1995) (and other SI containing proteins, such as 30S particles and Qbeta replicase (Brown et al., 1995)), phenylalanyl-tRNA synthetase (Peterson et al., 1993; Peterson et al., 1994), autoimmune antibodies that recognize RNA (Tsai et al., 1992), E2F transcription factor (Ishizaki et al., 1996) and various HIV associated proteins (Tuerk et al., 1993a; Giver et al., 1993; Tuerk et al., 1993b; Allen et al., 1995).

Aptamer selections have also been performed with proteins that were not known to bind to nucleic acids. Thrombin was one of the first candidates and its highest affinity aptamers were shown to be able to block thrombin's ability to cleave fibrinogen to fibrin (Bock et al., 1992; Kubik et al., 1994). Selections have also been carried out on many classes of proteins, including; growth factors (nerve growth factor (Binkley et al., 1995), basic fibroblast growth factor (Jellinek et al., 1993) and vascular endothelial growth factor (Jellinek et al., 1994)), antibodies (antibodies that bind to nuclear antigens (Tsai et al., 1992), insulin receptor (Doudna et al., 1995) and IgE class antibodies (Wiegand et al., 1996)), small molecules (cyanocobalamin (Lorsch et al., 1994), theophylline (Jenison et al., 1994), ATP (Sassanfer et al., 1993), GDP/GMP (Connell et al., 1994), chloroaromatics (Bruno et al., 1997), S-adenosyl methionine (Burke et al., 1997) and a panel of dyes (Ellingtion et al., 1990)) and a variety of other proteins (human thyroid stimulating hormone (Lin et al., 1996), human elastase (Bless et al., 1997), L-selectin (Hicke et al., 1996), protein kinase C (Conrad et al., 1994), Taq DNA polymerase (Dang et al., 1996) and reverse transcriptases, including; AMV (Chen et al., 1994), MMLV (Chen et al., 1994) and FIV (Chen et al., 1996).

The fact that one can generate novel nucleic acid ligands to a large variety of proteins led to the use of aptamers as an alternative to monoclonal and polyclonal antibody production for therapeutic and diagnostic uses. Diagnostic approaches using aptamers in place of antibodies have been evaluated. Aptamers to DNA polymerases have been used in hot start PCR to detect low copy number of the desired amplicon (Lin et al., 1997). Using an aptamer to block polymerase activity at low temperatures in PCR minimizes artifactual amplification and increases PCR sensitivity. Aptamers have also been used as a tool in assay development. An aptamer to neutrophil elastase was fluorescein labeled and used in a flow cytometry assay to determine elastase concentrations (Davis et al., 1996). The same aptamer was also used in an in vivo diagnostic imaging model of an inflamed rat lung (Charlton et al., 1997). Another aptamer to reactive green 19 (RG19) was also fluorescein labeled and used in a semi-quantitative bioassay for RG19 (Kawazoe et al., 1997). An immuno-assay using an aptamer detection reagent was also developed using a fluorescein labeled aptamer to VEGF (Drolet et al., 1996). The indirect immunoassay format was used for the quantitation of VEGF protein using a fluorescent substrate detection system.

In the enzyme-linked oligonucleotide assay (ELONA), one or more of the antibody reagents is replaced with an oligonucleotide sequence which specifically binds to the antigen. A specifically binding oligonucleotide sequence can be obtained by the in vitro selection of nucleic acid molecules which specifically bind to a target molecule using, for example, the SELEX method developed by L. Gold et al. (See Drolet, 1996). U.S. Pat. No. 5,472,841; U.S. Pat. No. 5,580,737; U.S. Pat. No. 5,660,985; U.S. Pat. No. 5,683,867; U.S. Pat. No. 5,476,766; U.S. Pat. No. 5,496,938; U.S. Pat. No. 5,527,894 U.S. Pat. No. 5,595,877; U.S. Pat. No. 5,637,461; U.S. Pat. No. 5,696,248; U.S. Pat. No. 5,670,637; U.S. Pat. No. 5,654,151; U.S. Pat. No. 5,693,502; U.S. Pat. No. 5,668,264; U.S. Pat. No. 5,674,685; U.S. Pat. No. 5,712,375; U.S. Pat. No. 5,688,935; U.S. Pat. No. 5,705,337; U.S. Pat. No. 5,622,828; U.S. Pat. No. 5,641,629; U.S. Pat. No. 5,629,155; U.S. Pat. No. 5,686,592; U.S. Pat. No. 5,637,459; U.S. Pat. No. 5,503,978; U.S. Pat. No. 5,587,468; U.S. Pat. No. 5,637,682; U.S. Pat. No. 5,648,214; U.S. Pat. No. 5,567,588; U.S. Pat. No. 5,707,796; U.S. Pat. No. 5,635,615; etc. WO 96/40991 and WO 97/38134 describe enzyme-linked oligonucleotide assays in which the capture antibody or the detecting antibody of a sandwich assay is replaced with a nucleic acid ligand. Generally, detection of the antigen:capture molecule complex is accomplished using a conventional enzyme-linked detecting antibody. Labeling of the oligonucleotide with a reporter enzyme, however, requires additional chemical synthesis steps and additional labor, difficulties also associated with assays which use antibody reagents as described above.

WO 96/40991 and WO 97/38134 also mention an embodiment in which the detection system is PCR amplification of a nucleic acid ligand which is part of the capture molecule:target molecule:detector molecule complex. These references suggest that the PCR primers used for amplification may contain reporter molecules such as enzymes, biotins, etc. Simple PCR amplification of a nucleic acid ligand provides additional quantities of the ligand, but has the disadvantage of requiring further separation steps to distinguish between the amplified ligand of interest and amplified nucleic acid impurities and primer dimers. Traditional gel separation requires intensive manual labor. Further, replicate experiments are required for statistical analysis and require additional time and labor. These problems exist for both DNA ligands and RNA ligands used in these oligonucleotide assays. The use of labeled primers allows detection of the PCR product, but does not overcome the problems of impurity and primer dimer amplification and is, therefore, not quantitative.

Dodge et al (US patent application 20020051974) describe the combination of real-time PCR quantification with immuno-PCR. In one aspect, the sample is exposed to an immobilized capture antibody (or other binding molecule) which captures the specific analyte molecule from the sample. The captured analyte molecule is then exposed to a detection reagent comprising an analyte-binding molecule (such as an antibody) conjugated to a nucleic acid. The bound detection molecule is then quantified using the real time PCR method described above. In this way, it was possible to use the sensitivity and dynamic range of real time PCR with the ease and specificity of immunoPCR.

This combination of real time PCR quantification and immuno-PCR is outlined in FIG. 1. A capture molecule (Cap A) is immobilized onto a solid surface via a linker X, which is cleavable in some embodiments. The sample is added and incubated, allowing the target protein for (Targ A) that Cap A recognizes to be captured specifically. After washing, the detection molecule (Detec A) is added. The detection molecule comprises two different parts. A target-binding portion (such as an antibody) and a nucleic acid tag (Tag A) such as a DNA oligonucleotide, which is attached via a linker (Linker Y), which may be cleavable in some embodiments.

After incubation, a sandwich complex is formed between the capture molecule (Cap A), the target (Targ A) and the detection molecule (Detec A). The surface is then washed to remove non-captured detection molecules. Then, the detection molecules are eluted and quantified using real time PCR.

The main limitation to the method of Dodge is that it measures a single analyte molecule. For many analytical applications, it is desirable to measure multiple different target molecules in a sample. For example, to understand a biological pathway, or how perturbations affect such a pathway, it may be important to quantify the amounts of many different molecules in the pathway, and the way these amounts change as a result of perturbations in the system.

Several methods for measuring multiple non-nucleic acid analytes in a pathway have been described. There are at least three main types of configurations for these: (i) two-dimensional (2D) array, (ii) encoded particle arrays (“liquid arrays”), and (iii) homogeneous solution-based arrays.

(i) Two-dimensional arrays for the measurement and quantification of proteins and other molecules are described in detail in the following US Patents, incorporated in their entirety herein by reference.
U.S. Pat. No. 5,837,551 Binding Assay
U.S. Pat. No. 6,475,808 Arrays of proteins and methods of use thereof
U.S. Pat. No. 6,365,418 Arrays of protein-capture agents and methods of use thereof.

The following articles also describe the construction and use of antibody microarrays for quantifying multiple analytes in parallel:
Ekins R, Chu F, Biggart E (1990): Multispot, multianalyte, immunoassay. Ann Biol Clin (Paris): 48:655-666.
Jenkins R E, Pennington S R (2001): Arrays for protein expression profiling: Towards a viable alternative to two-dimensional gel electrophoresis? Electrophoresis 1: 13-29.

2D arrays of capture agents, usually antibodies, can measure multiple analytes in parallel. Each feature on the array is derivatized with a single specificity, and the location of the each such specificity is known. By incubating samples with such an array, the amount of binding of analyte molecules to the array can be determined. Because the specificity of the different features is known, one can then infer the identity and, by extension, the amount of the different analytes in the sample according to there binding occurs on the array. 2D arrays can be used in in which the amount of analyte binding is directly measured, but this usually requires the sample to be labeled before being applied to the array. This type of approach has been reported by Haab et al (2001) and Miller et al (2003).

Alternatively, one can use a sandwich assay approach, as discussed above. It the case of arrays, however, there are multiple different capture agents, each one at a different location (on different features) of the array. The detection agents for each target are labeled and mixed together to form a “cocktail” of detection agents. When this cocktail is applied to the capture agent array that has been exposed to sample, the detection agents bind to the captured analytes on the features of the array. The resulting signal indicates the presence and/or amount of the analytes, as determined by the feature location.

(ii) Encoded particle arrays are analogous to the 2D arrays, except that the individual capture agent specificities are immobilized onto micropartiles. Each particle also contains an identification code such as a fluorescent signature (Kettman et al, 1998) or a bar code (Nicewarner-Pena et al, 2001), etc. The different encoded particles with attached capture agent can be mixed with the sample, allowing the analytes to be captured on the particles. Then, the amount of captured analyte on each particle is determined, as well as the identity (and therefore binding specificity) of each particle. By inference, one can determine the presence and/or amount of the different analytes that were present in the sample. As with 2D arrays, one can detect bound analyte either using sample labeling or by using a cocktail of detection agents.

(iii) homogeneous solution based arrays are described in the following publications and use the eTag™ system from Aclara (www.aclara.com):
J P Miller, (2002) Analytical & Research Technology. Meeting Today's Analytical Demands of Systems Biology. 3 (1): 16-17.
M Fogarty, (2002) Future Drug Discovery. Systems Biology and Beyond. 2 (1): Supplement.
J P Miller, (2002) The Scientist. Bridging Genomics and Proteomics. 16 (15):35.

All of the abovementioned systems for the parallel quantification of multiple proteins suffer from limitations. For the 2D arrays, difficulties arise in the preparation of reproducible microspots of capture reagent. Methods for delivering small volumes of protein do not result in even spot size and density and activity of the spotted capture molecule. Also, the sensitivity of the 2D arrays or the bead-based systems is limited since it is not generally possible to use amplification methods for detection. In particular, it is not possible to take advantage of the almost limitless amplification ability of nucleic acids. Finally, none of the systems mentioned above can measure the presence of cell surface markers with a large dynamic range. Of the three configurations mentioned above, only the homogenous solution-based methods can accurately quantitate cell surface marker amounts, but it is limited in terms of sensitivity and dynamic range.

For this reason, there is still the need for a method that allows for the measurement of multiple molecules in a sample that is highly sensitive, easy to use, and requires very small amounts of sample.

Throughout this application various publications (including patents and patent applications) are referenced. The disclosures of these publications in their entireties are hereby incorporated by reference.

### SUMMARY OF THE INVENTION

Accordingly, one object of the present invention is to provide a quantitative method for detecting the amounts of a plurality of different target molecules in a sample where the method has equal or improved sensitivity to conventional methods, has improved dynamic range, has improved resistance to contamination, has improved detection and which requires fewer human manipulation steps.

In one embodiment, the invention provides methods for quantitating or detecting the presence of a plurality of different target molecules in a sample, comprising: (a) exposing a sample, which contains or is suspected of containing the target molecules, to one or more different capture molecules capable of binding to the target molecules to form capture molecule:target molecule complexes; (b) adding to the capture molecule:target molecule complexes a plurality of detector molecules, each detector molecule being capable of specifically binding to a target molecule to form a capture molecule:target molecule:detector molecule ternary complex, wherein each detector molecule comprises a unique nucleic acid sequence tag which is different from the tag on another detector molecule; (c) separating the detector molecules in said ternary complexes from the unbound detector molecules; (d) dividing the detector molecules in the ternary complexes of step (c) into a plurality of samples; (e) performing real time PCR on each of the plurality of samples of step (d), wherein each PCR reaction has PCR primers specific for one or more nucleic acid sequence tags on the detector molecules; and (f) analyzing real time PCR data to determine the presence or quantity of the detector molecules and the corresponding target molecules present in the sample.

In some embodiments, the method further comprises a step of washing the capture molecule:target molecule complexes to remove unbound sample after step (a). In other embodiments, the step (c) described above is performed by washing the capture molecule:target molecule:detector molecule complexes to remove the unbound detector molecules.

In some embodiments, the capture molecules are immobilized to a solid support during step (a) or (b). In other embodiments, the capture molecules are in solution during step (a) or (b), and are immobilized to a solid support after step (b). In other embodiments, the capture molecules comprise linkers for immobilization to a solid support. In still other embodiments, the capture molecules are labeled with biotin and are bound to a streptavidin or avidin-coated support. In some embodiments, a plurality of capture molecules are used. In other embodiments, a single capture molecule is used and wherein the capture molecule is capable of specifically capturing more than one target molecules. In some embodiments, the capture molecules are antibodies.

In some embodiments, the target molecules are proteins or fragments thereof. In some embodiments, the target molecules are cytokines selected from the group consisting of growth hormone, insulin-like growth factors, human growth hormone, N-methionyl human growth hormone, bovine growth hormone, parathyroid hormone, thyroxine, insulin, proinsulin, relaxin, prorelaxin, glycoprotein hormones, follicle stimulating hormone (FSH), thyroid stimulating hormone (TSH), leutinizing hormone (LH), hematopoietic growth factor, vesicular endothelial growth factor (VEGF), hepatic growth factor, fibroblast growth factor, prolactin, placental lactogen, tumor necrosis factor-alpha, tumor necrosis factor-beta, mullerian-inhibiting substance, mouse gonadotropin-associated peptide, inhibin, activin, vascular endothelial growth factor, integrin, nerve growth factors (NGFs), NGF-beta, platelet-growth factor, transforming growth factors (TGFs), TGF-alpha, TGF-beta, insulin-like growth factor-I, insulin-like growth factor-II, erythropoietin (EPO), osteoinductive factors, interferons, interferon-alpha, interferon-beta, interferon-gamma, colony stimulating factors (CSFs), macrophage-CSF (M-CSF), granulocyte-macrophage-CSF (GM-CSF), granulocyte-CSF (G-CSF), thrombopoietin (TPO), interleukins (ILs), IL-1, IL-1alpha, IL-2, IL-3, IL-4, IL-5, IL-6, IL-7, IL-8, IL-9, IL-11, IL-12, LIF, SCF, neurturin (NTN) and kit-ligand (KL).

In some embodiments, the capture molecules bind the target molecules regardless of phosphorylation of the target molecules and each detector molecule specifically binds to a target molecule phosphorylated at a specific site within the target molecule, such that specifically phosphorylated target molecules are quantitated or detected.

In other embodiments, capture molecules specifically bind to target molecules that are phosphorylated at a specific site and the detector molecules bind to the target molecules regardless of the phosphorylation of the target molecules, such that phosphorylated target molecules are quantitated or detected.

In still other embodiments, the capture molecules bind to a moiety that is present on a subset of target molecules, and the detector molecules bind to the target molecules regardless of the presence of the moiety. The moiety may be phosphotyrosine, ubiquitin, a sumo protein, or a form of glycosylation.

The objects of the invention will become apparent in the course of the following description of exemplary embodiments that can be achieved by the present method for detecting the presence of a plurality of target molecules in a sample that may contain the target molecules, where the method uses the following steps:
(a) exposing a sample, which may contain or is suspected of containing the target molecules, to a one or a plurality of different capture molecules capable of binding to the target molecules to form capture molecule:target molecule complexes, where the number of target molecules that can be specifically captured by the capture molecules is greater than one;
(b) adding to the capture molecule:target molecule complexes a plurality of detector molecules, each detector molecule being capable of specifically binding to a target molecule to form a capture molecule:target molecule: detection molecule ternary complex, wherein each detection molecule also contains a unique nucleic acid sequence tag, such that each detection molecule contains a nucleic acid sequence tag different from that on other detection molecules, and
(c) separating the detection molecules in the said ternary complexes from those that are not in such ternary complexes to form a sample enriched with detection molecules that formed ternary complexes, and
(d) dividing the sample enriched with detection molecules that formed ternary complexes into a plurality of enriched samples, and
(e) performing real time PCR on each of the plurality of enriched samples, wherein each PCR reaction has PCR primers specific for one or a few nucleic acid sequence tags on the detection molecules, and where these PCR primers amplify only a subset of the nucleic acid tags on the detection molecules, such that more than one PCR reaction must be performed in order to quantify all of the nucleic acids in the plurality of nucleic acids attached to the plurality of detection molecules from step (b), and
(f) analyzing the real time PCR data to determine the presence or amounts of the target molecules present in the sample from step (a).

In this way, each PCR reaction detects the presence of a single detection molecule specificity, the presence of which in the sample enriched with detection molecules that formed ternary complexes is correlated with the presence of the target molecules that formed the ternary complexes. In the simplest case, there are n targets being interrogated, n capture molecules in the plurality of capture molecules and n detection molecules in the plurality of detection molecules, where n is some integer greater than one. In this case, step (d) would consist of dividing the sample enriched with detection molecules that formed ternary complexes into n samples, each of which would be analyzed by real time PCR using a single primer set that is specific for one of the nucleic acid sequence tags. Preferably, 3×(n) PCR reactions would be performed, as described except that each one would be run in triplicate for statistical reasons.

Alternatively, by using multicolor probes in real time PCR reactions, it is possible to detect 2, 3, 4 or more different nucleic acid sequence tags in a single PCR reaction containing 2, 3, 4 or more different primer sets for the sequence tags and using probes with different sequences, respectively. This form of multiplexing would reduce the total number of real time PCR reactions that would need to be run in order to measure the n different target molecules.

The method mentioned above uses the concept of a sandwich assay, in which the target molecules are each “sandwiched” between their cognate capture molecules and detection molecules. In one embodiment, the function of the capture molecules is to cause the target molecules to be immobilized onto the surface so that the now immobilized target molecules can cause immobilization, and thereafter enrichment, of detection molecules that bind to the target molecules. An alternative method differs in that there is no capture molecule. Rather, the target molecules are immobilized onto a surface in a less specific manner. For example, in cases where the target molecules are proteins and the sample is a biological fluid or liquid extract, the proteins thus contained may be non-specifically adsorbed to a surface through physical adsorption. Once immobilized, the detection of such immobilized target molecules is identical to that described above in the case of the sandwich assays.

Accordingly, in another embodiment, the invention provides methods for quantitating or detecting the presence of a plurality of different target molecules in a sample, comprising: (a) adding to an immobilized sample suspected of containing target molecules a plurality of detector molecules, each detector molecule being capable of specifically binding to a target molecule to form a target molecule:detector molecule complex, wherein each detector molecule comprises a unique nucleic acid sequence tag which is different from the tag on other detector molecule; (b) washing the surface to remove the non-immobilized detector molecules; (c) eluting the immobilized detector molecules; (d) dividing the detector molecules eluted in step (c) into a plurality of samples; (e) performing real time PCR on each of the plurality of samples in step (d), wherein each PCR reaction has PCR primers specific for one or more nucleic acid sequence tags on the detector molecules; and (f) analyzing the real time PCR data to determine the presence or amounts of the detector molecules and the corresponding target molecules present in the sample.

In some embodiments, the sample in step (a) contains a cell. In some embodiments, the target molecules are on the surface of the cell. In some embodiments, the immobilized sample in step (a) is tissue. In some embodiments, the sample is immobilized by covalent coupling to the surface. In other embodiments, the sample is immobilized by non-covalent attachment to the surface.

In another embodiment, the invention also provides methods for quantitating or detecting the presence of a plurality of target molecules on surface of a cell, comprising: (a) adding to a cell a plurality of detector molecules, each detector molecule being capable of specifically binding to a target molecule to form a target molecule:detector molecule complex, wherein each detector molecule comprises a unique nucleic acid sequence tag which is different from the tag on other detector molecule; (b) separating the detector molecules that formed complexes with the target molecules on the cell from unbound detector molecules; (c) dividing the detector molecules that formed complexes with the target molecules on the cell of step (b) into a plurality of samples; (d) performing real time PCR on each of the plurality of samples of step (c), wherein each PCR reaction has PCR primers specific for one or more nucleic acid sequence tags on the detector molecules; and (e) analyzing real time PCR data to determine the presence or amounts of the detector molecules and the corresponding target molecules present on the surface of the cell.

In some embodiments, the cells are enriched based on the presence of a cell surface marker before step (a). In some embodiments, the cells are enriched using a surface coated with a binding molecule that specifically binds to the cell surface marker. In some embodiments, the surface is a surface from a well of a microtiter plate. In some embodiments, the surface is a surface of a bead or a particle. In some embodiments, different cells are enriched using different surface coated with different binding molecules that specifically bind to different cell surface markers.

In some embodiments, the sample to be analyzed consists of cells. In order to determine the presence and quantity of proteins on cell surfaces, the cells are first immobilized onto a surface or onto beads or particles. A plurality of detection molecules, as above, are then added and allowed to bind to the cell surface target molecules, if present, according to the binding specificities of the individual detection molecules. After washing away the non-bound detection molecules, the bound detection molecules are eluted and analyzed by real time PCR as above. In this context, there are at least two different ways to immobilize the cells onto the surface, beads or particles. In the first case, the interaction is non-specific and takes advantage of the ability of cells to bind to certain surfaces, such as those coated with polylysine, fibronectin, etc. In the second case, certain cells in a sample are specifically captured onto the surface by capture molecules that bind to specific cell surface factors. In this aspect of the present invention, the capture molecules will purify a certain subset of cells in a sample of cells by selectively immobilizing them. The detection of target molecules on the surface of such captured cells is then performed as above. In essence, this is a sandwich assay, as described in the first case, above, except that the sandwiched target is a cell rather than a target molecule.

In any of the embodiments described herein, the detector molecules may be DNA-labeled antibodies. In some embodiments, the DNA are linked to the antibodies via linkers.

In any of the embodiments described herein, the sample may be selected from the group consisting of blood, serum, plasma, sputum, urine, semen, cerebrospinal fluid, sinovial fluid, bronchial aspirate and aqueous extracts from tissues or cells.

In any of the embodiments described herein, the detection of PCR product in the real time PCR reaction is performed using non-primer probes capable of binding to each nucleic acid tag on the capture molecules, wherein each non-primer probe comprises a nucleic acid having one or more fluorescent dye labels. In some embodiments, the nucleic acid of each non-primer probe comprises two fluorescent dye labels, a reporter dye and a quencher dye, which fluoresce at different wavelengths. In some embodiments, the nucleic acid of each non-primer probe comprises two labels, a reporter fluorescent dye and a non-fluorescent quencher molecule. In some embodiments, the nucleic acid tags on the detector molecules are RNA and the RNA nucleic acid tags are reverse transcribed to form DNA before or during amplifying step (e). In some embodiments, a single nucleic acid sequence tag is analyzed by real time PCR in each real time PCR reaction. In other embodiments, more than one nucleic acid sequence tag is analyzed by real time PCR in each real time PCR reaction by using different sequence-specific detection probes with different spectrally distinguishable signals.

The methods of the invention are improvements over other multiplexed immunoassay platforms for the following reasons: the dynamic range and sensitivity are higher due to the immense amplification potential of nucleic acids; there is no need to prepare microscopic features derivatized with protein capture molecules, a process that is very difficult to achieve reproducibly; the equipment and procedures used are very similar to those performed in many laboratories and does not require unusual equipment or training.

The invention also provides kits for quantitating or detecting a plurality of target molecules in a sample comprising one or more capture molecules; a plurality of detector molecules, wherein each detector molecule comprises a binding factor and a unique nucleic acid tag; and an instruction for performing any of the methods described herein. The kits may further comprise PCR primers specific for nucleic acid tags of the detector molecules, labeled non-primer probes for quantification during PCR. In some embodiments, the PCR primers can also be used as PCR primers for detecting mRNA encoding the target proteins.

### BRIEF DESCRIPTION OF THE DRAWINGS

FIG. 1. The principle of real time immuo-PCR in a non-multiplexed fashion.

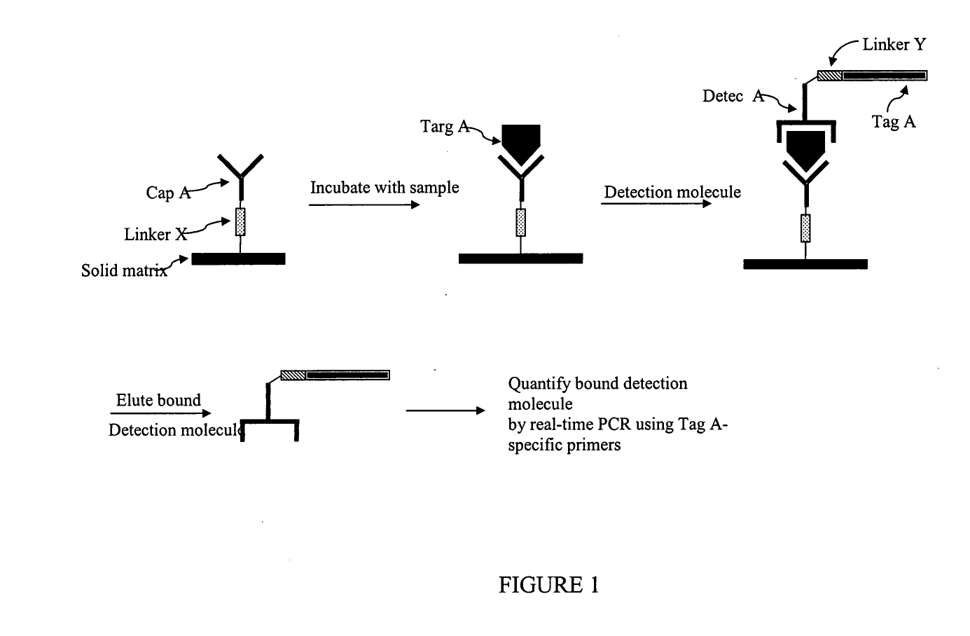

FIG. 2. Multiplexed real time immuno-PCR using a sandwich approach.

FIG. 3. Multiplexed real time immuno-PCR without a sandwich.

FIG. 4. Analysis of cell surface markers on a particular cell population from a sample containing multiple cell types.

FIG. 5. Standard curves derived from immuno-real time PCR assays for IL-5, IL-6 and TNF-α

### DETAILED DESCRIPTION OF THE PREFERRED EMBODIMENTS

The present invention provides a method for quantitating or detecting the presence of a plurality of target molecules in a sample which may contain the target molecules, comprising: (a) exposing a sample, which may contain or is suspected of containing the target molecules, to a one or a plurality of different capture molecules capable of binding to the target molecules to form capture molecule:target molecule complexes, where the number of different target molecules that can be specifically captured by the capture molecules is greater than one; (b) adding to the capture molecule:target molecule complexes a plurality of detector molecules, each detector molecule being capable of specifically binding to a target molecule to form a capture molecule:target molecule:detection molecule ternary complex, wherein each detection molecule also contains a unique nucleic acid sequence tag, such that each detection molecule contains a nucleic acid sequence tag different from that on any other detection molecule; (c) separating the detection molecules in the said ternary complexes from those that are not in such ternary complexes to form a sample enriched with detection molecules that formed ternary complexes; (d) dividing the sample enriched with detection molecules that formed ternary complexes into a plurality of enriched samples; (e) performing real time PCR on each of the plurality of enriched samples, wherein each PCR reaction has PCR primers specific for one or a few nucleic acid sequence tags on the detection molecules, and where each set of PCR primers amplifies only one sequence tag, and only a subset of the nucleic acid tags on the detection molecules will be amplified, if present in the sample, in each such PCR reaction, such that more than one PCR reaction must be performed in order to quantify all of the nucleic acids in the plurality of nucleic acids attached to the plurality of detection molecules from step (b); and (f) analyzing the real time PCR data to determine the presence or amounts of the detection molecules and, by extension, the corresponding target molecules present in the sample.

The method may further comprise washing the capture molecule:target molecule complexes to remove unbound sample after step (a). Alternatively, the method may further comprise washing the capture molecule:target molecule:detector molecule complexes to remove unbound detector molecules after step (b).

In one method of the invention, the presence of target molecules in a sample which may contain the target molecule are detected and may be quantitated by exposing the sample which may contain the target molecules to a plurality of capture molecules capable of binding the target molecules to form capture molecule:target molecule complexes. The presence of the target molecules in a sample can be determined without further quantitating the amount of the target in the sample, if detection only is desired. Detection is achieved by observing a detectable signal from the signal resulting from PCR amplification of the sequence tags present on the detection molecules. Quantitation is achieved by comparing the sample to standard samples with known amounts of target compounds.

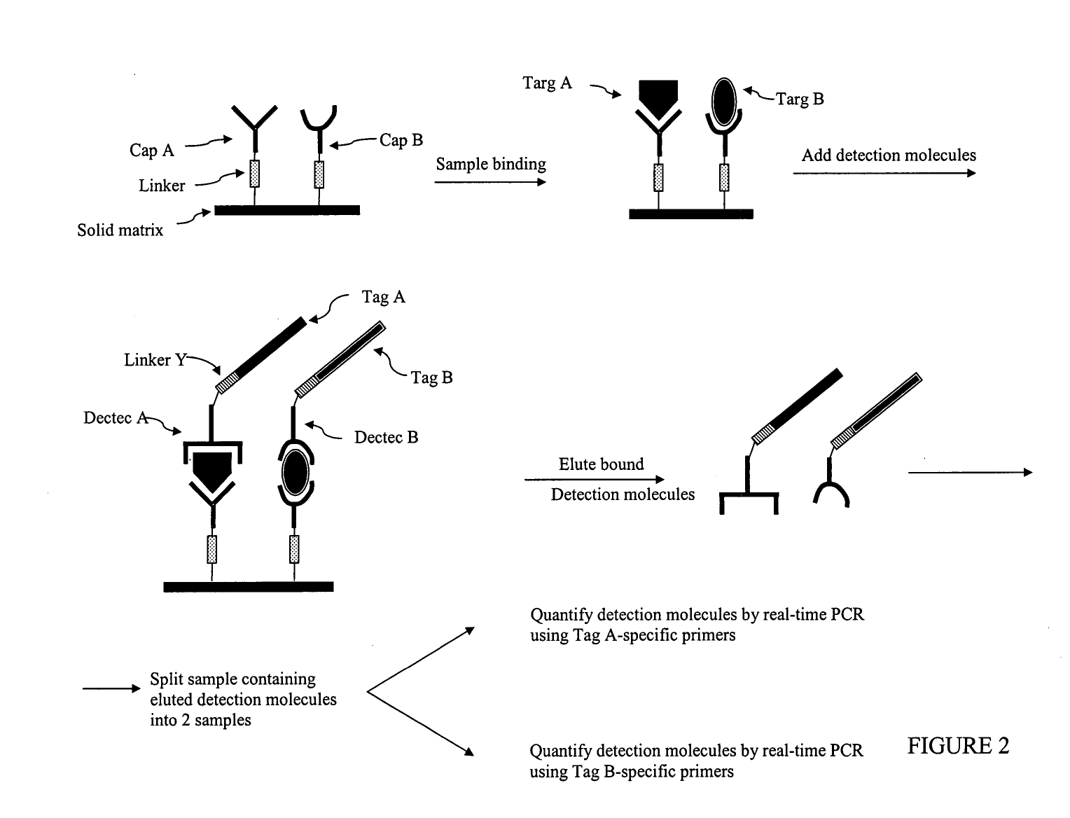

This concept is illustrated in FIG. 2. In the figure, two target molecules are measured in parallel. It should be understood that this multiplexing of 2 is used for simplicity of illustration and multiplexing of much larger numbers is possible with this method. First, a surface is prepared with 2 different capture molecules, Cap A and Cap B. A sample is added, and Cap A captures target A according to its target-binding capacity. Likewise, Cap B captures target B. After washing, a plurality of detection molecules are added, in this case two, Detec A and B, which have attached nucleic acid tags (Tag A and Tag B, respectively). After incubation and washing, the detection molecules are eluted to form an “elution sample.” The elution sample is then divided into two separate samples, one of which is analyzed by real time PCR using primers specific for Tag A, and the other is analyzed by real time PCR using primers specific for Tag B. These real time PCR measurements provide information about Detec A and B, respectively, in the elution sample, which in turn provides information about the amounts of Targ A and Targ B in the original sample.

The term “capture molecule” as used herein means any molecule or target binding fragment thereof capable of specifically binding to the target molecule so as to form a capture molecule:target molecule complex. In this context, “specifically binding” means that the capture molecule binds to the target molecule based on recognition of a binding region or epitope on the target molecule. The capture molecule preferably recognizes and binds to the target molecule with a higher binding affinity than it binds to other molecules in the sample. Preferably, the capture molecule uniquely recognizes and binds to the target molecule.

Typically, the capture molecule is an antibody, preferably a monoclonal antibody or an affinity-purified polyclonal antibody, which immunologically binds to the target molecule at a specific determinant or epitope. The term “antibody” is used in the broadest sense and specifically covers monoclonal antibodies as well as antibody fragments (e.g., Fab, Fab′, F(ab′)₂, scFv, Fv diabodies and linear antibodies), so long as they exhibit the desired binding activity. For a review of sFv see (Pluckthun, 1994). Diabodies are described more fully in, for example, EP 404,097; WO 93/11161; and Hollinger et al., 1993). Linear antibodies are described in Zapata et al (1995). The capture molecule may also be fusion proteins comprising an antibody or fragment thereof, or any other modified configuration of the immunoglobulin molecule that comprises an antigen recognition site.

The preparation of monoclonal antibodies specific for a target molecule is well known and described, for example, in (Harlow and Lane, eds., 1988). The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by (Kohler & Milstein, 1975) or may be made by recombinant DNA methods (see, e.g. U.S. Pat. No. 4,816,567 (Cabilly et al.)). Recombinant antibodies herein specified include those made by conventional approaches (Kohler & Milstein, 1975), phage display (Winter et al, 1994; Smith, 1985), mRNA display (Liu, 2000), ribosome display (Schaffitzel et al, 1999, 2001), microbial display (Daugherty et al, 1999; Francisco et al, 1993; Wittrup, 2001; Feldhaus et al, 2003) and any other selection method generally known to those skilled in the art of combinatorial biology (Szostak, 1992; Roberts and Ja, 1999). Antibodies and antibody fragments may be displayed on the surface of a filamentous bacteriophage as described in U.S. Pat. No. 5,750,373, for example and the references cited therein. See also EP 844306; U.S. Pat. No. 5,702,892; U.S. Pat. No. 5,658,727; WO 97/09436; U.S. Pat. No. 5,723,287; U.S. Pat. No. 5,565,332; and U.S. Pat. No. 5,733,743.

Polyclonal antibodies may also be used in the present invention. Preferably such antibodies will be affinity purified against the antigen. Description of methods for making polyclonal antibodies and affinity purifying them are given in Goding, J W (Monoclonal Antibodies: Principles and Practice, 3rd ed. San Diego: Academic Press; 1996).

The monoclonal antibodies herein specifically include “chimeric” antibodies (U.S. Pat. No. 4,816,567 (Cabilly et al.); and (Morrison et al., 1984); and humanized antibodies (Jones et al., 1986); (Reichmann et al., 1988); and (Presta, 1992). Alternatively, various artificial proteins known as “antibody mimetics” or “alternative scaffold”-based proteins that bind specifically to target molecules may also be substituted (Skerra, 2000; Xu et al, 2002).

The capture or detection molecules may also be a high-affinity nucleic acid ligand which binds to the target molecule, e.g. an aptamer. The term “nucleic acid ligand” as used herein means a nucleic acid, including naturally occurring and non-naturally occurring nucleic acids, having a specific binding affinity for the target molecule. Nucleic acid ligands may be identified and prepared using the SELEX method described in U.S. Pat. No. 5,270,163; U.S. Pat. No. 5,475,096; U.S. Pat. No. 5,496,938; WO 96/40991; and WO 97/38134, for example. The nucleic acid ligand may be DNA or RNA.

The capture molecules may also be binding proteins, receptor or extracellular domains (ECD) thereof capable of forming a binding complex with a ligand, typically a polypeptide or glycopeptide ligand. In one embodiment, the binding protein is a cytokine superfamily receptor or receptor ECD and the target molecule is a cytokine. “Cytokine superfamily receptors”, which can be used as the capture molecule, are a group of closely related glycoprotein cell surface receptors that share considerable homology including frequently a WSXWS domain and are generally classified as members of the cytokine receptor superfamily (see e.g. (Nicola et al., 1991) and (Skoda. et al., 1993)). Generally, these receptors are interleukins (IL) or colony-stimulating factors (CSF). Members of the superfamily include, but are not limited to, receptors for: IL-2 (beta and gamma chains) (Hatakeyama et al., 1989); (Takeshita et al., 1991); IL-3 (Itoh et al., 1990); (Gorman et al., 1990); Kitamura et al., 1991 a); (Kitamnura et al., 1991b); IL-4 (Mosley et al., 1989); IL-5 (Takaki et al., 1990); (Tavernier et al., 1991); IL-6 (Yamasaki et al., 1988); (Hibi et al., 1990); IL-7 (Goodwin et al., 1990); IL-9 (Renault et al., 1992); granulocyte-macrophage colony-stimulating factor (GM-CSF) (Gearing et al., 1991); (Hayashida et al., 1990) granulocyte colony-stimulating factor (G-CSF) (Fukunaga et al., 1990a); (Fukunaga et al., 1990b); (Larsen et al., 1990); EPO (D'Andrea et al., 1989); (Jones et al., 1990) Leukemia inhibitory factor (LIF) (Gearing et al., 1991); oncostatin M (OSM) (Rose et al., 1991); and also receptors for prolactin (Boutin et al., 1988); (Edery et al., 1989); growth hormone (GH) (Leung et al., 1987); ciliary neurotrophic factor (CNTF) (Davis et al., 1991); c-Mpl (M. Souyri et al., 1990); (I. Vigon et al., 1992).

The capture molecules may further comprise a linker for attaching to a solid support. A “linker” is a molecule comprising two or more functional groups that are capable of joining two molecules and/or provides space between two molecules and flexibility of the two molecules. For attaching to a solid support, the surface of the solid support may be derivatized with a chemical functional group (e.g., amino group, carboxy group, oxo group, thiol group) to react with one functional group of the linker. Any linkers that are well known in the art may be used, e.g., homo- or hetero-bifunctional linkers (for example, see 2003-2004 Applications Handbook and Catalog, Pierce). A linker may include a peptide, protein, oligonucleotide, lipid, sugar, polyethylene glycol, cholesterol, fusion protein, bispecific antibody, or crosslinking agent. Cleavable linkers may be used and may provide advantage to elute capture molecule:target molecule:detection molecule ternary complexes from a surface that the capture molecule is immobilized to. Examples of cleavable linkers are chemically cleavable linker (such as linkers having esters, disulfide bonds, bonds that can be broken by oxidation), enzymatically cleavable linker (such as bonds that can be hydrolyzed by an enzyme), and photocleavable linker.

The “detector molecule” (interchangeably termed “detection molecule” herein) comprises two components: a binding factor and a nucleic acid tag. The binding factor can be any specific binding agent (e.g., antibody, antibody fragment, and nucleic acid ligand), including all of those listed above as potential “capture molecules”. The detector molecule may a DNA-labeled antibody. In the detector molecule, the link between the binding factor and the nucleic acid tag may be made in a number of ways known to one skilled in the art of bioconjugation chemistry. Any linkers described herein or that are known in the art may be used. For example, techniques known for use in conventional immuno-PCR, for example, crosslinking with Sulfo-SMCC (Pierce, Rockford, Ill.) may be used. The bond between the binding factor and the nucleic acid tag is preferably covalent, but also may be non-covalent if the non-covalent bond is sufficiently stable to remain intact during the course of the assay.

The nucleic acid tag of the detector molecule described herein comprises a sequence specific for each detector molecule for detecting a specific target molecule, such that specific primers and/or specific non-primer probes may be designed for each detector molecule in order to assay a plurality of detector molecules at the same time using real time PCR.

The binding factor of a detector molecule may be a nucleic acid ligand. The term “nucleic acid ligand” as used herein means a nucleic acid, including naturally occurring and non-naturally occurring nucleic acids, having a specific binding affinity for the target molecule. Nucleic acid ligands may be identified and prepared using the SELEX method described in U.S. Pat. No. 5,270,163; U.S. Pat. No. 5,475,096; U.S. Pat. No. 5,496,938; WO 96/40991; and WO 97/38134, for example.

The “target molecule” may be any 3-dimensional chemical compound that binds to the capture molecule. The target molecule will generally be a peptide, a protein, carbohydrate or lipid derived from a biological source such as bacterial, fungal, viral, plant or animal samples. By “target molecule,” we also include multimolecular species such as oligomeric proteins, viruses, and cells. The samples may include blood, plasma, serum, sputum, urine, semen, cerebrospinal fluid, sinovial fluid, bronchial aspirate, organ tissues, and aqueous extracts from tissues or cells, etc. Additionally, however, the target molecule may be a smaller organic compound such as a drug, drug-metabolite, dye or other small molecule present in the sample. Preferably, the small molecule is an organic target molecule having a molecular weight of about at least 100 and up to about 1,000 grams/mole, more preferably about 200 to about 700 grams/mole. When small molecules are the target molecule, it is preferred to use nucleic ligands as the capture molecule. A preferred group of target molecules are cytokines. “Cytokine” is a generic term for proteins released by one cell population which act on another cell as intercellular mediators. Examples of such cytokines are lymphokines, monokines, and traditional polypeptide hormones. Included among the cytokines are growth hormone, insulin-like growth factors, human growth hormone (hGH), N-methionyl human growth hormone, bovine growth hormone, parathyroid hormone, thyroxine, insulin, proinsulin, relaxin, ptorelaxin, glycoprotein hormones such as follicle stimulating hormone (FSH), thyroid stimulating hormone (TSH), and leutinizing hormone (LH), hematopoietic growth factor, hepatic growth factor (HGF), fibroblast growth factor, prolactin, placental lactogen, tumor necrosis factor-alpha (TNF-alpha and TNF-beta), mullerian-inhibiting substance, mouse gonadotropin-associated peptide, inhibin, activin, vascular endothelial growth factor (VEGF), integrin, nerve growth factors such as NGF-beta, platelet-growth factor, transforming growth factors (TGFs) such as TGF-alpha and TGF-beta, insulin-like growth factor-I and -II, erythropoietin (EPO), osteoinductive factors, interferons such as interferon-alpha,-beta, and -gamma, colony stimulating factors (CSFs) such as macrophage-CSF (M-CSF), granulocyte-macrophage-CSF (GM-CSF), and granulocyte-CSF (G-CSF), thrombopoietin (TPO), interleukins (IL's) such as IL-1, IL-1alpha, IL-2, IL-3, IL-4, IL-5, IL-6, IL-7, IL-8, IL-9, IL-11, IL-12 and other polypetide factors including neurturin (NTN), LIF, SCF, and kit-ligand. As used herein the foregoing terms are meant to include proteins from natural sources or from recombinant cell culture. Similarly, the terms are intended to include biologically active equivalents; e.g., differing in amino acid sequence by one or more amino acids or in type or extent of glycosylation.

The capture molecule may be attached to a solid support before, during or after forming the capture molecule:target molecule complex. Specific capture molecules, e.g. antibodies or aptamers, are prepared as described above and purified using conventional separation techniques. The capture molecules are then attached to solid supports using passive absorbance or other conventional (e.g., chemical) techniques for attaching proteins to solid supports. In a preferred embodiment, the solid support is coated with one member of a known binding pair, e.g. streptavidin, and the capture molecule is labeled with the other member of the binding pair, e.g. biotin. The biotin labeled capture molecule:target molecule complex or the capture molecule:target molecule:detector molecule ternary complex may be formed in solution phase and later captured by the streptavidin-coated or avidin-coated support. In a particularly preferred form of this embodiment, the support is a streptavidin coated tube and the detector molecule is a DNA-labeled antibody.

Other suitable binding pairs which can be used in this embodiment include any known epitope tags and binding partners therefore, generally antibodies which recognize the tag. The term “epitope tagged” refers to a capture molecule fused to an “epitope tag”. The epitope tag polypeptide has enough residues to provide an epitope against which an antibody thereagainst can be made, yet is short enough such that it does not interfere with activity of the capture molecule. The epitope tag preferably is sufficiently unique so that the antibody thereagainst does not substantially cross-react with other epitopes. Suitable tag polypeptides generally have at least 6 amino acid residues and usually between about 8-50 amino acid residues (preferably between about 9-30 residues). Examples include the flu HA tag polypeptide and its antibody 12CA5 (Field et al. Mol. Cell. Biol. 8:2159-2165 (1988)); the c-myc tag and the 8F9, 3C7, 6E10, G4, B7 and 9E10 antibodies thereto (Evan et al., Mol. Cell. Biol. 5(12):3610-3616 (1985)); and the Herpes Simplex virus glycoprotein D (gD) tag and its antibody (Paborsky et al, Protein Engineering 3(6):547-553 (1990)), digoxigenin/anti-digoxigenin antibody, FITC/anti-FITC antibody, His6/Ni columns, Protein A/antibody Fc regions, etc. In certain embodiments, the epitope tag is a “salvage receptor binding epitope”. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

The present invention teaches a method for detecting a plurality of molecules in parallel, i.e. multiplexed detection. The capture molecules used in an experiment thus must be capable of specifically binding to a plurality of molecules. In a preferred embodiment, this is achieved by providing a plurality of different capture molecules that are co-incubated with the sample. In such a case, the different capture molecules are generally mixed together before being put in contact with the sample. In cases where the capture molecule(s) are immobilized onto a surface or particles prior to being put into contact with the sample, a preferred method for accomplishing this is the following set of steps: a) the plurality of capture molecules are mixed together; b) the mixture is immobilized onto the surface; c) unbound capture molecules are removed by washing; d) the plurality of now immobilized capture molecules are exposed to the sample, as described above.

In cases where the capture molecule(s) are immobilized onto particles or beads prior to being put into contact with the sample, a preferred method for accomplishing this is the following set of steps: a) the plurality of capture molecules are mixed together; b) the mixture is immobilized onto the beads or particles; c) unbound capture molecules are removed by washing; d) the plurality of now immobilized capture molecules are exposed to the sample, as described above. In an alternative method, the following steps are used: a) each of the plurality of capture molecules are individually immobilized onto the beads or particles; b) unbound capture molecules are removed by washing; c) the different beads with different capture molecules are mixed together; d) the plurality of now immobilized capture molecules are exposed to the sample, as described above. In this method, it will also be appreciated that steps b) and c) may be reversed in order.

As mentioned above, the present invention teaches a method for detecting a plurality of molecules in parallel, so the capture molecules used in an experiment thus must be capable of specifically binding to a plurality of molecules. In the embodiment outlined above, this is achieved by providing a plurality of different capture molecules that are co-incubated with the sample. Another method for creating a capture molecule composition that is capable of specifically binding to a plurality of target molecules is to use a single capture molecule that specifically binds to a certain subset of molecules that may be present in the sample. For example, in a sample, there may be a number of molecules that share a molecular determinant (e.g., a moiety, a phosphorylation site) that can be specifically bound by the capture molecule. As an example, the capture molecule could be an antibody that specifically binds to phosphotyrosine. When this capture molecule is exposed to a sample containing multiple proteins, and a subset of these proteins contain phosphotyrosine, the capture molecule will specifically bind to this aforementioned subset at the expense of molecules without the phosphotyrosine moiety. An assay utilizing a capture molecule such as this and a sample containing proteins that may contain phosphotyrosine can be used to detect or quantitate a plurality of different phosphotyrosine-containing proteins in a sample. In this case, the detection molecules in the plurality of detection molecules would contain individual detection molecules, each of which could specifically bind to one protein that may be modified by phosphotyrosine. In this manner, the amounts of multiple phosphor-tyrosine-containing proteins could be measured in a sample in parallel. This approach may also be utilized to monitor other protein modifications as well. Examples include ubiquitinylated proteins, sumoylated proteins, proteins modified by conjugation to various lipids or fatty acids, proteins containing certain glycosylation moieties, and so on.

Another embodiment of the present invention will allow for one to measure the degree and/or location to which several different modifications have occurred to a single protein or other molecule. For example, some or all of the capture molecules used bind to target proteins regardless of the phorsphorylation of these proteins, and some or all of the detector molecules used preferentially bind to the target proteins that are phosphorylated at specific sites within the proteins, such that if the proteins in the sample are phosphorylated at the specific site, a ternary complex can be formed. By way of illustration but not limitation, this invention discloses a method for measuring the presence of phosphoserine at 5 different locations in protein X, and simultaneously measures the amount of ubiquitinylation and phosphotyrosine modification of the same protein. In this theoretical example, protein X is 700 amino acids in length and has serines that may be phosphorylated at positions 100, 200, 300, 400 and 500. In this case, the capture molecule is a molecule that can specifically bind to protein X regardless of its modification state. The sample is incubated with the immobilized capture molecule so that the modified and unmodified protein X molecules are captured. The surface is then washed. A plurality of detection molecules is then prepared, comprising the following detection molecules:

1. A detection molecule that binds to protein X regardless of its modification state.

2. A detection molecule that binds to the segment of protein X between amino acid positions 90-110, but only when this segment is not modified by phosphorylation.

3. A detection molecule that binds to the segment of protein X between amino acid positions 90-110, but only when this segment is modified by phosphorylation.

4. A detection molecule that binds to the segment of protein X between amino acid positions 190-210, but only when this segment is not modified by phosphorylation.

5. A detection molecule that binds to the segment of protein X between amino acid positions 190-210, but only when this segment is modified by phosphorylation.

6. A detection molecule that binds to the segment of protein X between amino acid positions 290-310, but only when this segment is not modified by phosphorylation.

7. A detection molecule that binds to the segment of protein X between amino acid positions 290-310, but only when this segment is modified by phosphorylation.

8. A detection molecule that binds to the segment of protein X between amino acid positions 390-410, but only when this segment is not modified by phosphorylation.

9. A detection molecule that binds to the segment of protein X between amino acid positions 390-410, but only when this segment is modified by phosphorylation.

10. A detection molecule that binds to the segment of protein X between amino acid positions 490-510, but only when this segment is not modified by phosphorylation.

11. A detection molecule that binds to the segment of protein X between amino acid positions 490-510, but only when this segment is modified by phosphorylation.

12. A detection molecule that binds to ubiquitin, including ubiquitin that is bound to proteins.

13. A detection molecule that binds to phosphotyrosine.

As described herein, each of these 13 detection molecules comprises, in addition to the binding factor, a unique nucleic acid tag sequence allowing for detection by real time PCR.

After incubation of the immobilized sample with the plurality of 13 detection molecules, the surface is washed and then bound detection molecules are then eluted from the surface. The sample is then divided into 13 samples. Each of the 13 samples is subjected (in triplicate) to a real time PCR analysis of the amount of a single of the nucleic acid sequence tags, thus providing information regarding the following:

1. The reaction using PCR primers specific for the detection molecule that binds to protein X regardless of its modification state provides a measure of the total amount of protein X in the sample (modified and unmodified forms).

2. The reaction using PCR primers specific to the detection molecule that binds the segment of protein X between amino acid positions 90-110, but only when this segment is not modified by phosphorylation, provides a measure of the amount of protein X in the sample that is not modified by phosphorylation at position 100.

3. The reaction using PCR primers specific to the detection molecule that binds the segment of protein X between amino acid positions 90-110, but only when this segment is modified by phosphorylation, provides a measure of the amount of protein X in the sample that is modified by phosphorylation at position 100.

4. The reaction using PCR primers specific to the detection molecule that binds the segment of protein X between amino acid positions 190-210, but only when this segment is not modified by phosphorylation, provides a measure of the amount of protein X in the sample that is not modified by phosphorylation at position 200.

5. The reaction using PCR primers specific to the detection molecule that binds the segment of protein X between amino acid positions 190-210, but only when this segment is modified by phosphorylation, provides a measure of the amount of protein X in the sample that is modified by phosphorylation at position 200.

6. The reaction using PCR primers specific to the detection molecule that binds the segment of protein X between amino acid positions 290-310, but only when this segment is not modified by phosphorylation, provides a measure of the amount of protein X in the sample that is not modified by phosphorylation at position 200.

7. The reaction using PCR primers specific to the detection molecule that binds the segment of protein X between amino acid positions 290-310, but only when this segment is modified by phosphorylation, provides a measure of the amount of protein X in the sample that is modified by phosphorylation at position 200.

8. The reaction using PCR primers specific to the detection molecule that binds the segment of protein X between amino acid positions 390-410, but only when this segment is not modified by phosphorylation, provides a measure of the amount of protein X in the sample that is not modified by phosphorylation at position 400.

9. The reaction using PCR primers specific to the detection molecule that binds the segment of protein X between amino acid positions 390-410, but only when this segment is modified by phosphorylation, provides a measure of the amount of protein X in the sample that is modified by phosphorylation at position 400.

10. The reaction using PCR primers specific to the detection molecule that binds the segment of protein X between amino acid positions 490-510, but only when this segment is not modified by phosphorylation, provides a measure of the amount of protein X in the sample that is not modified by phosphorylation at position 500.

11. The reaction using PCR primers specific to the detection molecule that binds the segment of protein X between amino acid positions 490-510, but only when this segment is modified by phosphorylation, provides a measure of the amount of protein X in the sample that is modified by phosphorylation at position 500.

12. The reaction using PCR primers specific to the detection molecule that binds to ubiquitin, including ubiquitin that is bound to proteins, provides a measure of the amount of protein X in the sample that is modified by ubiquitinylation.

13. The reaction using PCR primers specific to the detection molecule that binds to phosphotyrosine provides a measure of the amount of protein X in the sample that contains phosphotyrosine.

Any suitable solid support is useful in the method of the present invention. Suitable solid supports include membranes, charged paper, nylon, beads, polystryrene ELISA plates, PCR tubes (Numata et al, 1997), V-bottom polycarbonate plates (Chang et al, 1997), etc. Suitable membranes include nitrocellulose membranes and polyvinylidine difluoride membranes. In a preferred embodiment, the capture molecule is bound to a polymer bead, tube or plate, for example a conventional polycarbonate plate.

The capture molecule or molecules (alone or attached to the solid support) are exposed to a sample which may contain the target molecule under conditions suitable for complex formation and, if target molecule is present, the capture molecules binds to the target molecules forming capture molecule:target molecule complexes. To avoid nonspecific binding of the target molecule to a solid support, the solid support is generally treated to block nonspecific binding sites prior to exposing the sample to the capture molecule. Common blocking agents include dilute protein solutions (about 3-5%), for example bovine serum albumin (BSA), and nonionic detergents (polyvinyl pyrrolidone, PVP-40) and TWEEN 20. Typically, the capture molecules bound to the solid support are incubated under conditions sufficient to block nonspecific binding sites. In direct binding, blocking occurs by incubating the solid support having the capture molecules bound thereto in a solution of the blocking agent at about room temperature (or other suitable temperature) for several hours (2-20 hours) with agitation according to known methods. The blocking solution is then generally washed from the solid support to remove remaining blocking agent.

The sample to be tested for the presence of the target molecules is then placed in contact with the capture molecule under conditions sufficient to allow the formation of a capture molecule:target molecule complexes. Optionally, the sample may be diluted as needed prior to contact with the capture molecules. In most cases, the target sample will be an aqueous sample, although other sample media are suitable as long as the media allows formation of the desired binding complex. Ordinary optimization of assay parameters is within the skill of the practitioner in this field and will generally involve optimizing ionic strength, divalent metal ion concentration, pH, etc. Optimization is generally performed for each different type of sample and composition of capture molecules.

After a time sufficient to allow formation of the capture molecule:target molecule complexes, unbound or remaining sample is removed from the complexes, generally by washing. Typically, the complex is washed with 1-10 volumes of water or suitable buffer.

In some embodiments of the present invention, a chemical crosslinking step is performed after the capture molecules have formed complexes with the target molecules and the non-bound sample molecules have been removed. A chemical crosslinking, preferably capable of proceeding quickly, is performed to stabilize the capture molecule:target molecule complexes. After the formation of cross-links between the capture molecules and target molecules, the surfaces are thoroughly washed to remove unreacted crosslinking reagent. In addition, the crosslinking reagents are quenched, if possible. Following this step, the assay will be performed as described above. Optionally, for certain applications, after the crosslinking steps, the captured proteins may be partially or completely denatured by the addition of denaturants such as chaotropic agents or ionic detergents. After denaturation, the denaturants are removed. Then, the plurality of detection molecules is added. In this way, detection molecules that recognize denatured but not folded targets will be capable of functioning. There are many type of chemical crosslinking that can be used, in particular the photo-crosslinking described in Fancy et al (2000) may be preferred.

In all of the embodiments, the detection molecules in the capture molecule:target molecule:detection molecule ternary complexes may be eluted from the surface before being divided into a plurality of samples and then being analyzed by real time PCR. There are many methods for achieving this. One is the use of high (e.g., pH 11) or low (e.g. pH 2.5) pH. In such cases, after elution, the solution may be neutralized before proceeding to the next steps in the analysis. Alternatively, heat may be used to elute. Alternatively, cleavage of ternary complex from the surface, bead or particle may be used to elute the detection molecules. This method has the added advantage that, while the specifically immobilized detection molecules will thus be released, many of the non-specifically surface-bound detection molecules, if any, may not be efficiently released by such a procedure. This may increase the specific signal to non-specific noise ratio and thus improve the sensitivity of the multiplexed assay. To achieve specific cleavage of the ternary complexes from the surface, bead or particle, the linkage between the capture molecule and the surface, bead or particle must be engineered to contain a bond that can be cleaved at will. Examples of cleavable links are chemically cleavable links (esters, disulfide bonds, bonds broken by oxidation) enzymatically cleavable links (bonds that can be hydrolyzed by an enzyme), and photocleavable links. The most preferred embodiments use cleavable links that can be cleaved without significant changes in the chemical or physical changes in the buffer. The photocleavable group may be most appropriate. One non-limiting example of an appropriate photocleavable linking group is a (nitrophenyl)-ethyl moiety, which can be readily cleaved with a 300-360 nm light pulse. An example of a method for immobilizing capture molecules onto a surface via a photocleavable linking group is to first biotinylate the antibodies with a compound that has a photocleavable group between the biotin moiety and the antibody-reactive moiety. After conjugation, the antibodies thus modified are applied to a surface coated with a biotin-binding protein such as avidin or streptavidin. They can later be cleaved from the surface by treatment with light of the appropriate wavelength to cleave the photocleavable link. An example of a photocleavable biotin compound for this type of purpose is EZ-Link NHS-PC-LC-Biotin, available from Peirce.

Alternatively, elution of the nucleic acid tag from the remainder of the detection molecule may be used, but this is less preferred because it may not have a beneficial effect on the signal:noise ratio.

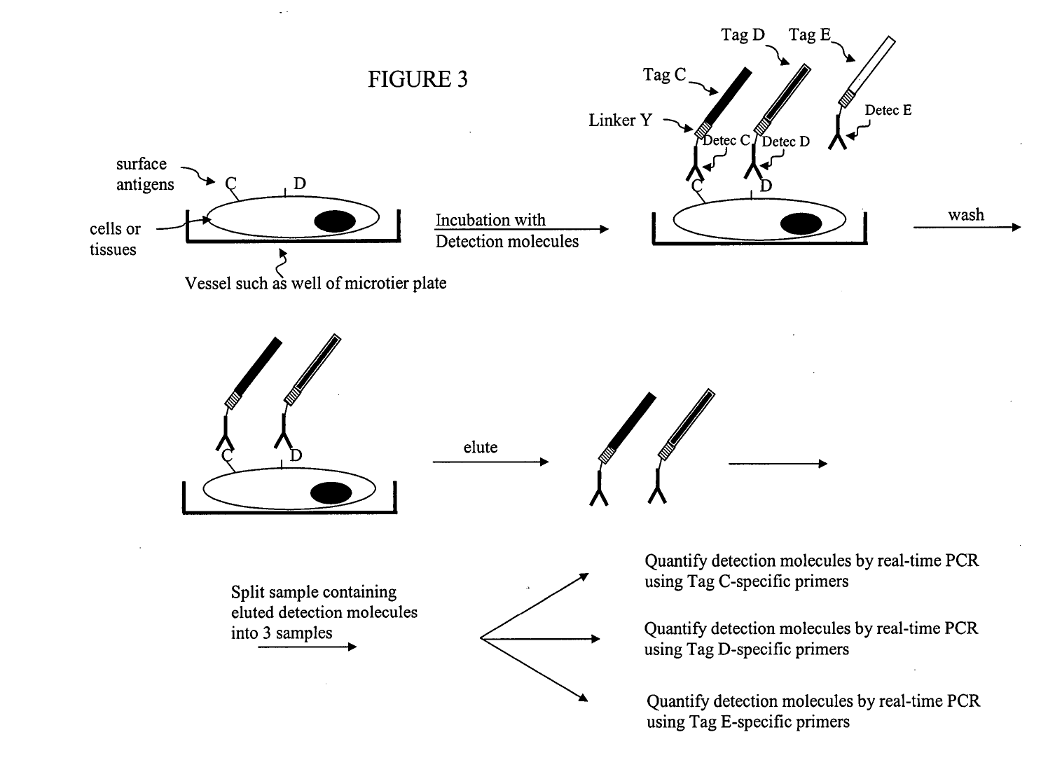

All of the cases mentioned above in this section rely on the use of a sandwich assay, in which the target molecules are each “sandwiched” between their cognate capture molecules and detection molecules. In some of these embodiments, the function of the capture molecules is to cause the target molecules to be immobilized onto the surface so that the now immobilized target molecules can cause immobilization, and thereafter enrichment, of detection molecules that bind to the target molecules. An alternative aspect differs in that no capture molecule is used. For example, a component (such as a cell, a tissue) of a sample that contains or is suspected of containing the target molecules is immobilized (e.g., covalently or non-covalently) onto a surface (such as a beads, a particle, a tube) and the target molecules are detected by detector molecules as described herein. The target molecules are immobilized onto a surface in a less specific manner. This is illustrated in FIG. 3. The figure shows cells attached to a solid surface, such as the well of a microtiter plate. Other examples are other biological samples that have been immobilized onto a surface by physical adsorption or chemical coupling, for example. In the figure, the immobilized cells (only one cell is shown) have antigens C and D on its surface. These are target proteins for the assay and their abundance on the cells is of interest. A plurality of detection molecules (2 are shown) is added, as before. In this example, two of the detection molecules (Detec C and Detec D) bind to the surface because their cognate targets (antigens C and D) are present; Detec E does not bind because its target is not present. After washing, the detection molecules are eluted to form an “elution sample.” The elution sample is then divided into three separate samples, each of which is analyzed by real time PCR to determine the abundance of a single tag sequence, as described for FIG. 2.

In cases where the target molecules are proteins and the sample is a biological fluid or liquid extract, the proteins thus contained may be non-specifically adsorbed to a surface through physical adsorption. Examples of surfaces that bind proteins non-specifically and can therefore be used for this type of immobilization are plastics (especially polystyrene, such as those supplied by Nunc), nitrocellulose, aminopropylsilane-coated glass, and polylysine-coated glass. Once immobilized, the detection of such immobilized target molecules is identical to that described above in the case of the sandwich assays. Another way of non-specifically immobilizing proteins in a sample onto a surface is to use a surface with reactive groups that can chemically couple to the proteins. There are numerous examples of such chemical groups known to those of common skill in the art, including N-hydroxysuccinimide, epoxides, and so on.

In another aspect of the present invention, the sample to be analyzed includes cells. In order to determine the presence and quantity of proteins on cell surfaces, the cells may be immobilized onto a surface or onto beads or particles. A plurality of detection molecules, as above, are added and allowed to bind to the cell surface target molecules, if present, according to the binding specificities of the individual detection molecules. After washing away the non-bound detection molecules, the bound detection molecules are eluted and analyzed by real time PCR as above. In this context, there are at least two different ways to immobilize the cells onto the surface, beads or particles. In the first case, the interaction is non-specific and take advantage of the ability of cells to bind to certain surfaces, such as those coated with poly-lysine, fibronectin, or others known to one of common skill in the art. This is the case shown in FIG. 3, as described above.

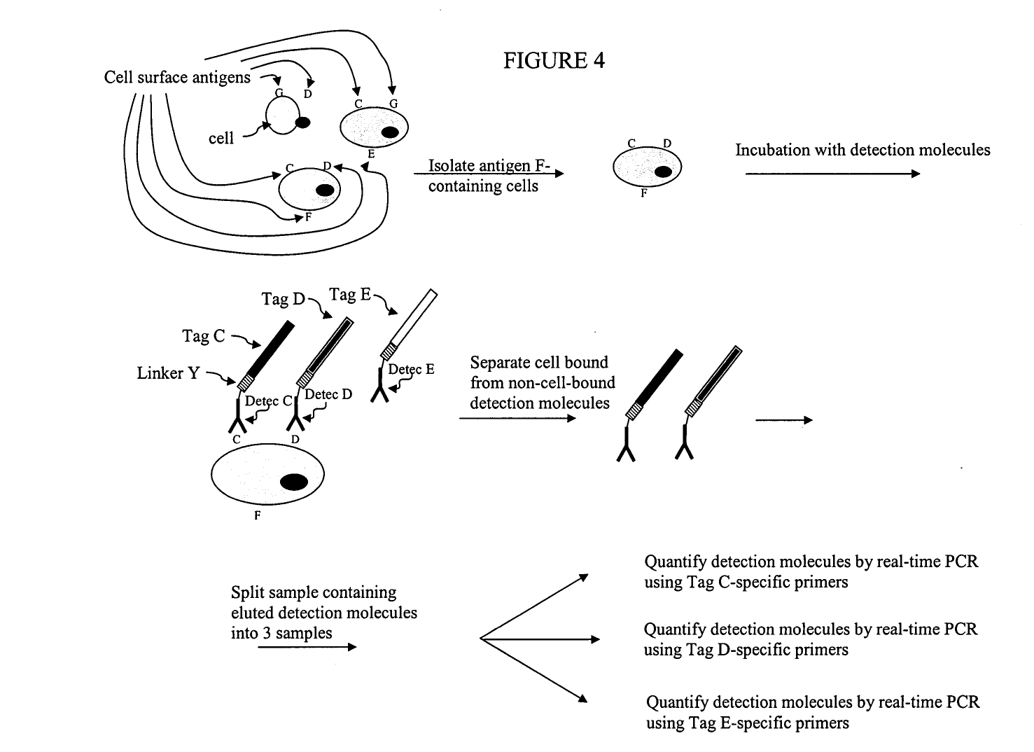

The cells in the sample may be enriched by separating cells based on the presence of a cell surface marker. Certain cells in a sample are specifically captured onto the surface by capture molecules that bind to specific cell surface molecules. In this aspect of the present invention, the capture molecules will purify a certain subset of cells in a sample of cells by selectively immobilizing them. The detection of target molecules on the surface of such captured cells is then performed as above. In essence, this is a sandwich assay, as described in the first case, above, except that the sandwiched target is a cell rather than a target molecule. This is illustrated in FIG. 4. In this example, multiple cell types are present in the sample. An example of such a sample would be blood, which contains several different cell types. Different cell types have different markers, as shown. A particular sub-population of cells is purified from the other cell types, on the basis of possessing cell surface antigen F. There are several ways of accomplishing this separation, such as exposing the cells to a surface derivatized by an antibody that binds to antigen F; alternatively, beads derivatized with an antibody specific for antigen F may be used to purify cells with the antigen F on their surfaces; alternatively, fluorescence-activated cell-sorting (FACS) may be used in conjunction with a fluorescently labeled antibody that can bind to antigen F. Whatever the method, the purified cell population is exposed to the plurality of detection molecules, washed, and the amounts of the detection molecules are determined as described in FIG. 3. Another level of multiplexing in this assay is also possible but is not shown. A microtiter plate derivatized with different antibodies in different wells can be prepared. The sample (blood for instance) is added to each well, so that different wells purify different cell populations. The detection molecules are added and the other steps performed as described. In this way, it is possible to analyze the surface antigens on several different cell sub-populations in parallel from the same sample.

An example of the utility of this latter method would be to determine the presence and amounts of certain cell surface proteins on certain types of cells. One could prepare a microtiter plate that, in the first well, has and antibody against the cell surface molecule CD4 immobilized onto its surface. In a second well, an anti-CD8 antibody is immobilized. Blood from a patient is added to both wells. After incubation and washing, the first well will have captured CD4+ cells and the second well will have captured CD8+ cells. Into each well, a plurality of detection molecules is then added. By example, the detection molecules could detect CD antigens 1 through 10. After washing and elution of the detection molecules, real time PCR is used to analyze the presence of the different detection molecules in wells number one and two. These results would indicate the difference in expression levels of CD antigens 1 through 10 on CD4 versus CD8 cells. This is meant to illustrate the type of application that could be used by this approach, and could be expanded to include dozens or hundreds of different wells, each containing a different capture molecule and therefore purifying a different cell population. Such an approach could be used to detect and characterize even rare cells in the blood such as stem cells, cancer cells, fetal cells, and so on.

Regardless of the type of material to be analyzed, or whether sandwich or non-sandwich assay methods are used, the detector molecules are generally dissolved in an aqueous solution, preferably an aqueous buffer solution and contacted with the capture molecule:target molecule complexes. Suitable buffers are those well known in the art for buffering antibody and nucleic acid ligand molecules, and include known buffers used in conventional ELISA, PCR, immuno-PCR and ELONA assays. After a time sufficient to allow formation of the desired ternary complexes, unbound detector molecules are removed from the complexes, preferably by washing with buffer. The ternary complex is then ready for elution and amplification.

After elution of the detector molecules from the ternary complexes, the sample is divided into a plurality of samples, each of which is measured by real time PCR using a defined set of PCR primers, where different PCR reactions utilize different primers. Each PCR amplification is performed in the presence of a non-primer detectable probe which specifically binds the PCR amplification product, i.e., the amplified detector DNA moiety. PCR primers are designed according to known criteria and PCR may be conducted in commercially available instruments. The non-primer probe may comprise a nucleic acid having one or more fluorescent dye labels. The probe is preferably a DNA oligonucleotide specifically designed to bind to the amplified detector molecule. The probe may comprise two labels, one reporter dye and one quencher molecule. The probe preferably has a 5′ reporter dye and a downstream 3′ quencher molecule (including fluorescent or non-fluorescent) covalently bonded to the probe which allow fluorescent resonance energy transfer. The reporter dye and the quencher molecule may generate fluorescence at different wavelengths. Suitable fluorescent reporter dyes include 6-carboxy-fluorescein (FAM), tetrachloro-6-carboxy-fluorescein (TET), 2,7-dimethoxy-4,5-dichloro-6-carboxy-fluorescein (JOE), hexachloro-6-carboxy-fluorescein (HEX), VIC, Cy3, ROX, Texas Red, and Oregon Green. Suitable quencher molecules include 6-carboxy-tetramethyl-rhodamine (TAMRA), and Black Hole Quenchers. These dyes are commercially available from Perkin-Elmer, Philadelphia, Pa.; Applied Biosystems, Foster City, Calif.; and Qiagen, Valencia, Calif. Detection of the PCR amplification product may occur at each PCR amplification cycle. At any given cycle during the PCR amplification, the amount of PCR product is proportional to the initial number of template copies. The number of template copies is detectable by fluorescence of the reporter dye. When the probe is intact, the reporter dye is in proximity to the quencher molecule which suppresses the reporter fluorescence. During PCR, the DNA polymerase cleaves the probe in the 5′-3′ direction separating the reporter dye from the quencher molecule increasing the fluorescence of the reporter dye which is no longer in proximity to the quencher molecule. The increase in fluorescence is measured and is directly proportional to the amplification during PCR. See Heid et al, 1996. This detection system is now commercially available as the TaqMan.RTM. PCR system from Perkin-Elmer, which allows real time PCR detection.

In an alternative embodiment, the reporter dye and quencher molecule may be located on two separate probes which hybridize to the amplified PCR detector molecule in adjacent locations sufficiently close to allow the quencher molecule to quench the fluorescence signal of the reporter dye (de Silva et al., 1998; Rasmussen et al, 1998). As with the detection system described above, the 5′-3′ nuclease activity of the polymerase cleaves the one dye from the probe containing it, separating the reporter dye from the quencher molecule located on the adjacent probe preventing quenching of the reporter dye. As in the embodiment described above, detection of the PCR product is by measurement of the increase in fluorescence of the reporter dye.

After elution of the detector molecules from the ternary complexes, the sample may be divided into a number of samples that is less than the number of different detector molecules used, and thus the two or more real time PCR reactions are performed in each divided sample. To carry out multiple real time PCR reaction in one divided sample (e.g., one tube or well), the non-primer probes used in one sample have different nucleotide sequences and are specific for the corresponding nucleic acid tags of the detector molecules, and the reporter dye of each non-primer probe generates signals different from the reporter dye on the other non-primer probe used in the same divided sample. For example, FAM, TET, JOE, and HEX may be used as reporter dye for different non-primer probes and TAMRA may be used as quencher molecule for the probes.

The detector molecule nucleic acid tags may also be RNA oligonucleotides. In this case, the RNA is first reverse transcribed to DNA before PCR amplification (Gibson et al., 1996). It is possible to reverse transcribe an RNA detector molecule directly from the ternary complex. Preferably, the reverse transcription reaction is conducted at an elevated temperature, that is, a temperature sufficient to dissociate the RNA oligonucleotide detector molecule from the ternary complex. Reverse transcription is preferably conducted with avian myeloblastosis virus (AMV) reverse transcriptase since this transcriptase enzyme has been found to work sufficiently well at elevated temperatures required for dissociation of the RNA oligonucleotide from the ternary complex. Preferred temperatures at which the reverse transcription reaction is conducted are about 60° C. to about 70° C. AMV reverse transcriptase is commercially available, for example, from Promega, Madison, Wis. After reverse transcription to DNA, PCR amplification and detection may be performed as described above when the nucleic acid moiety is DNA. In one embodiment, reverse transcription and PCR amplification are conducted together in a single reaction (RT-PCR). In a preferred embodiment, real time PCR or real time RT-PCR described above are used to detect the PCR products.

In other embodiments of this invention, other real time PCR detection strategies may be used, including known techniques such as intercalating dyes (ethidium bromide) and other double stranded DNA-binding dyes used for detection (e.g. SYBR green, a highly sensitive fluorescent stain, FMC Bioproducts), dual fluorescent probes (Wittwer, C. et al., (1977) BioTechniques 22:130-138; Wittwer, C. et al., (1997) BioTechniques 22:176-181) and panhandle fluorescent probes (i.e. molecular beacons; Tyagi S., and Kramer F R. (1996) Nature Biotechnology 14:303-308). Although intercalating dyes and double stranded DNA binding dyes permit quantitation of PCR product accumulation in real time applications, they suffer from the previously mentioned lack of specificity, detecting primer dimer and any non-specific amplification product. Careful sample preparation and handling, as well as careful primer design, using known techniques should be practiced to minimize the presence of matrix and contaminant DNA and to prevent primer dimer formation. Appropriate PCR instrument analysis software and melting temperature analysis permit a means to extract specificity (Ririe, K., et al. (1977) Anal. Biochem. 245: 154-160) and may be used with these embodiments.

One of the preferred platforms for performing the real time PCR is the use of the Applied Biosystems 7900HT Micro Fluidic Card with the appropriate PCR machine from the same company.

The PCR method of the invention has a dynamic range which allows the detection of target molecules at concentrations from about 0.005 pg/mL to about 5000 pg/mL. The method is preferably used to detect target molecules at concentrations in the range of about 1 pg/mL to about 1000 pg/mL.

The compounds or materials used in the method of the invention can also be provided in the form of a kit. An assay kit will usually contain the capture molecules, optionally bonded to a solid support, beads or particles, including magnetic particles, and the detector molecules, and may contain one or more of the following: primers for PCR amplification of the nucleic acid moieties, a non-primer probe for quantification during PCR, calibration standards for a desired targets (calibration samples), control samples containing known amounts/concentrations of the desired targets, other PCR reagents used in the PCR steps (alternatively, these reagents can be purchased separately), PCR plates or tubes, optionally coated with a binding molecule, e.g. streptavidin or avidin, instructions for using the reagents and for performing any methods described herein, and components of the kit to quantify or simply to detect the targets in samples. The sequence of a PCT primer and the corresponding nucleic acid tag of a detector molecule for detecting a target protein may be designed that the PCR primer may also be used for detecting RNA encoding the target protein via PCR. Accordingly, the kits may be used for detecting both target protein and the mRNA encoding the target protein. The kits may be in a container with a label. Suitable containers include, e.g., bottles, vial, and test tubes. The containers may be formed from a variety of the materials, such as glass or plastic.

Utility

The methods and kits of the present invention can be used to detect multiple target (such as polypeptides) within a biological pathway, and to detect upregulation, downregulation, and/or quantitation of relative amounts of target molecules within a given system. Assaying multiple target molecules simultaneous are particularly important for drug screening, e.g., detecting perturbation of a biological pathway and efficacy of a treatment. The methods and the kits of the invention can also be used to detect modification of a target molecule, such as glycosylation and phosphorylation.

The assay of the present invention is useful for the detection of target molecules in clinical diagnosis of physiologic conditions in the same manner as ELISA, immuno-PCR and ELONA have been used conventionally. The assay may also be used to detect the presence of a target molecule in food, environmental, water, effluent, etc. samples.

Using the methods of the invention, detector molecules containing a nucleic acid moieties can be directly detected and quantitated across at least five logarithmic concentrations, to as low as a few hundred molecules. One advantage of this method over other multiplexed detection methods is that, after performing the binding and washing steps with the capture and detection molecules, but before real time PCR analysis, the sample can be saved indefinitely and further analyzed at a later date, since nucleic acids can be stored without significant degredations.

Without further description, it is believed that one of ordinary skill in the art can, using the preceding description and illustrative examples, make and utilize the present invention to the fullest extent. The following examples therefore specifically point out preferred embodiments of the present invention, and are not to be construed as limiting in any way of the remainder of the disclosure.

### EXAMPLES OF DETAILED METHODS

The following detailed protocols outline how certain embodiments of the invention can be performed.

Synthesis of Detection Molecules (Antibody-DNA Conjugates)

A single-strand DNA of 40-70 base pairs is synthesized with an amino group at the 5′ end. The DNA is coupled through its amino group to a bi-functional Linker Y, which has an amino-reacting group at one end and a sulfhydryl-reacting group at the other end, separated by a spacer. Examples of Linker Y are Sulfo-SMCC (sulfosuccinimidyl 4-[N-maleimidomethyl]-cy-clohexane-1-carboxylate) (Pierce, Rockford, Ill.). The coupled adduct is purified away from the un-reacted Linker Y by gel filtration chromatography using G-25 resin.

A reduced antibody is prepared by incubating the antibody in 50 mM DTT/50 mM sodium phosphate/S mM EDTA, pH 6.0, followed by gel filtration chromatography using 50 mM sodium/5 mM EDTA phosphate buffer, pH 7.0, as the mobile phase. The reduced antibody and the DNA-Linker Y adduct are mixed at a ratio of 1:5 and incubated at room temperature overnight. The DNA antibody conjugate is formed via the chemical reaction between the sulfhydryl group of the reduced antibody and the sulfhydryl-reacting group of Linker Y. The DNA-antibody conjugate is purified by anion exchange chromatography, followed by Protein A affinity purification to remove free DNA. The concentration of the antibody can be quantified by Bradford assay, and the concentration of the DNA can be quantified by its absorbance at 260 nm, or it can be quantified by using real time PCR.

Sandwich-Type Immunoassay

The wells of a microtiter plate, such as MaxiSorp (Nunc), are coated with 100 μl of the capture antibody mixture at 5 μg/ml (total) in 0.1 M sodium bicarbonate, pH 9.6, at 4 degree overnight. The wells are washed 3 times with 10 mM sodium phosphate/150 NaC/0.1% Tween 20, pH 7.4 (PBST), then blocked with 200 μl 4% non-fat milk (NFM) in PBS at room temperature for 1 hour. The wells are washed 3 times with PBST. One hundred μl of serially diluted antigen mixtures in 2% NFM/PBS are added to the wells in triplicate. A solution of NFM/PBS without antigen is used to measure background signal. The binding reactions are allowed to proceed at room temperature for 2 hours. After the incubation, the wells are washed 5 times with PBST, followed by the addition of 100 μl DNA-detecting antibody conjugate cocktail (5 nM each) in NFM/PBS and incubation at room temperature for 30 minutes. The wells are washed 7 times with PBST. The bound detecting antibody-DNA conjugates are eluted by incubation with 100 μl 0.1 M aqueous triethylamine for 20 minutes. The eluent is neutralized with 50 μl Tris-HCl (1 M, pH 8.0). The concentrations of the detecting antibody-DNA conjugates present in the eluent are quantified by real time PCR. Five pI of the eluent is added to the real time PCR reaction tubes, each containing one primer pair/probe specific to the DNA of each detecting antibody. The standard curve of each antigen is generated using a range of antigen concentrations in the immunoassays. The threshold cycle number (C_{T}) at each antigen concentration (y-axis) is plotted against antigen concentrations α-axis), and the data is fit to a curve using a regression program. The concentrations of the antigens in the test sample can be calculated using the experimentally derived standard curves.

Expression Profiling of Surface Antigens of Cultured Cells and Tissue Slices

Cells derived from cell lines or from primary cells are cultured as adhesion cells on 96-well culture plates. The cells are washed gently 3 times with ice cold PBS, and incubated with a cocktail of detection antibody-DNA conjugates (5 nM each) at 4 degree for 2 hours. The cells are then washed 7 times with ice cold PBS. Bound detection antibody-DNA conjugates are eluted with 0.1 M triethylamine and neutralized with Tris-HCl. The concentration of each detection antibody-DNA conjugate is determined by real time PCR as described above. The protocol for expression profiling of tissues is the same as described above except that tissue slices are immobilized on wells of the microtiter plate.

Real-Time PCR

Buffer A, nucleotides, ampli-Taq gold polymerase, and AmpErase uracil N-glycosylase (UNG) can be purchased from PE Applied Biosystems. 50 microliters/well of master mix (2.5 mM MgCl₂, 1× Buffer A, 0.5 uM upper primer, 0.5 uM lower primer, 40 nM probe, 200, uM of ATP, GTP, and CTP, 400 uM of UTP, 0.5 unit UNG, 1.5 units Taq polymerase) is added to the PCR wells in the 96 well PCR amplification plate (PE Applied Biosystems). The thermocycle conditions for the Taqman are, for example, 50 C for 3 min, 94 C for 12 min, and 40 cycles of 94 C for 15 s and 60 C for 1.5 min. Data is collected during the extension phase.

The threshold emission is calculated by the Sequence Detector 1.6 software (PE Applied Biosystems) as 10 standard deviations above the average increase in reporter dye emission due to the cleavage of the probe (ΔRn), of cycles 3 to 10. The C, value for each sample is determined by the software and exported to Softmax Pro (Molecular Devices, Sunnyvale, Calif.) for data reduction. The standard curve is fit using a four parameter non-linear regression.

### EXAMPLE

### Quantitation and Detection of Interleukin 5, Interleukin 6, and TNF-α Using Sandwich-type Immunoassay and Real-time PCR

Primer, Probe, and DNA Labels

DNA labels and primers were synthesized by using standard phosphoramidite chemistry. An amino group was incorporated at the 5′ end of the DNA labels through a C6 spacer during the DNA synthesis. To introduce a sulfhydryl through the reaction with the 5′ amino group of the DNA labels, the DNA labels were dissolved in PBS buffer (10 mM sodium phosphate, 150 mM NaCl, pH 7.4) and Sulfo-LC-SPDP (sulfosuccinimidyl 6-[3′-(2-pyridyldithio)-propionamido]hexanoate (Pierce Endogen, Rockford, Ill.) was added to the DNA solutions to a final concentration of 2 mM. The reactions were carried out at room temperature for 1 hour. The 2-pyridylthio group protecting the sulfhydryl was subsequently removed by 5.5 mM TCEP (Tris[2-carboxyethylphosphine]hydrochloride) (Pierce Endogen, Rockford, Ill.) at room temperature for 1 hour. The DNA labels with free sulfhydryl were purified by ethanol precipitation using 0.3 M sodium acetate. The sequences of the DNA labels and primers are:

Set A:

| | | |
|---|---|---|
| Set A: | | |
| DNA label: | 5′GCACTGCTTTCTACTCATCGGATGAGTTGTCATGTCCTGCGGAGAAGAAAGACGGAGAGT-3′ | (SEQ ID NO:1) |
| Upper primer: | 5′-GCACTGCTTTCTACTCATCG-3′ | (SEQ ID NO:2) |
| Lower primer: | 5′-ACTCTCCGTCTTTCTTCTCC-3′ | (SEQ ID NO:3) |
| Set B: | | |
| DNA label: | 5′CAATAACCACCCCTGACCGATGAGTTGTCATGTCCTGCGCATTCCTTCTTCTGGTCAG-3′ | (SEQ ID NO:4) |
| Upper primer: | 5′-CAATAACCACCCCTGACC-3′ | (SEQ ID NO:5) |
| Lower primer: | 5′-CTGACCAGAAGAAGGAATGC-3′ | (SEQ ID NO:6) |
| Set C: | | |
| DNA label: | 5′CCTACCAGACCAAGGTCAACCGATGAGTTGTCATGTCCTGCGGATCATTGCCCTGTGAGG-3′ | (SEQ ID NO:7) |
| Upper primer: | 5′-CCTACCAGACCAAGGTCAACC-3′ | (SEQ ID NO:8) |
| Lower primer: | 5′-CCTCACAGGGCAATGATCC-3′ | (SEQ ID NO:9) |

The sequences of upper/lower primer pairs of Set A, B, and C belong to the gene sequences of human interleukin 5, interleukin 6, and tumor necrosis factor-α (TNF-α), respectively. These primers can also be used to quantify the mRNA or cDNA of the corresponding genes by real time PCR. The sequence of the probe used was 5′-GATGAGTTGTCATGTCCTGC-3′ (SEQ ID NO:10). The probe was synthesized with the reporter dye FAM (6-carboxyfluorescein) at the 5′ end and the quencher molecule BHQ-1 (Black Hole Quencher-1) (Qiagen, Valencia, Calif.) at the 3′ end.

Synthesis of Detection Molecules (Antibody-DNA Conjugates)

The detection antibodies for human interleukin 5, interleukin 6, and TNF-α (BD Pharmingen, San Diego Calif.) were dissolved in PBS, pH 7.4. A 20 molar excess of the crosslinker LC-SMCC (succinimidyl-4-[N-maleimidomethyl]-cyclohexane-1-carboxy-[6-amidocaproate]) (Pierce Endogen, Rockford, Ill.), which reacted to the primary amines of the antibodies, was added and the mixtures were incubated for 2 hours at room temperature. The excess LC-SMCC was removed and the buffer in the reactions was exchanged into PBS, pH 7.0, using NAP-5 desalting columns (Amersham Biosciences, Piscataway, N.J.). The antibody solutions were concentrated to about 0.2 ml with Microcon YM-50 concentrators (Millipore, Bedford, Mass.). The DNA labels with free sulfhydryl groups were added to the LC-SMCC activated antibodies in a 12-fold molar excess and incubated for 2 hours at room temperature followed by incubation overnight at 4° C. The antibody-DNA conjugates were purified as follows: protein A columns were equilibrated with 1.5 M glycine, 250 mM NaCl, pH 8.3. The antibody-DNA reaction mixtures were loaded and the columns were washed with the equilibration buffer until the OD at 260 nm returned to baseline. The antibodies were eluted with 1.5 M glycine, 250 mM NaCl, 2.5 M MgCl₂, pH 8.3 and dialyzed into PBS.

Sandwich-Type Immunoassay

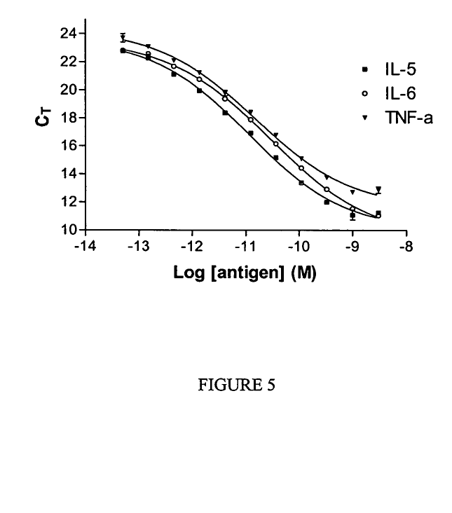

The wells of a 96-well microtiter plate (MaxiSorp, Nalge Nunc International, Rochester, N.Y.), were coated with 100 μl of the capture antibody mixture against interleukin 5, interleukin 6, and TNF-α (BD Pharmingen, San Diego Calif.) in 0.1 M sodium bicarbonate, pH 9.6, at 4° C. overnight. The concentration of each capture antibody in the mixture was 3 μg/ml. The wells were washed 3 times with 10 mM sodium phosphate/150 NaCl/0.1% Tween 20, pH 7.4 (PBST), then blocked with 200 μl 4% NFM in PBS at room temperature for 1 hour. The wells were washed 3 times with PBST. One hundred μl of serially diluted antigen mixtures in 50% adult bovine serum (ABS)/PBS were added to the wells in duplicate. A solution of ABS/PBS without antigen was used to measure background signal. The binding reactions were allowed to proceed at room temperature for 2 hours. After the incubation, the wells were washed 6 times with PBST, followed by the addition of 100 μl DNA-detecting antibody conjugate cocktail (5 nM each) in 2% NFM/PBS and incubation at room temperature for 40 minutes. The wells were washed 7 times with PBST. The bound detecting antibody-DNA conjugates were eluted by incubation with 100 μl 0.1 M aqueous triethylamine for 20 minutes. The eluent was neutralized with 150 μl of 0.33 M Tris-HCl, pH 8.0. The concentrations of the detecting antibody-DNA conjugates present in the eluents were quantified by real time PCR. Twenty μl of the eluent was added to the real time PCR reaction tubes, each containing one primer pair/probe set specific to the DNA label of each detecting antibody. The standard curve of each antigen was generated using a range of antigen concentrations in the immunoassays. The threshold cycle number (CT) at each antigen concentration (y-axis) was plotted against antigen concentrations α-axis), and the data were fit to a curve using a four-parameter logistic regression program (FIG. 5). The concentrations of the antigens in the test sample can be calculated using the experimentally derived standard curves.

Real-Time PCR

Nucleotides, buffer, AmpliTaq gold polymerase, and AmpErase uracil N-glycosylase (UNG) were purchased from PE Applied Biosystems (Foster City, Calif.). Each 50 uL reaction contained a mixture of 2.5 mM MgCl₂, 1× Buffer A, 0.45 uM upper primer, 0.45 uM lower primer, 250 nM probe, 200 uM of ATP, GTP, and CTP, 400 uM of UTP, 0.5 unit UNG, 2.5 units AmpliTaq gold polymerase, and the eluted samples from the immunoassay. The reaction mixtures were added to the PCR wells in the 96-well PCR amplification plate (Applied Biosystems, Foster City, Calif.). The thermocycle conditions for the real-time reactions were 50° C. for 2 min, 95° C. for 10 min, and 40 cycles of 95° C. for 15 s and 60° C. for 1 min. Data were collected during the extension phase.

Multiple real time PCR may be simultaneously carried out in one reaction vessel (tube or well). To carry out multiple real time PCR in one reaction vessel, the sequences of probes are different from each other and specific to each DNA-antibody conjugates, and are labeled with different reporter dyes such as 6-FAM, TET, HEX, Cy3, TAMRA, ROX, Texas Red, or Oregon Green at the 5′ end (Qiagen, Valencia, Calif.).

While the invention has necessarily been described in conjunction with preferred embodiments and specific working examples, one of ordinary skill, after reading the foregoing specification, will be able to effect various changes, substitutions of equivalents, and alterations to the subject matter set forth herein, without departing from the spirit and scope thereof. Hence, the invention can be practiced in ways other than those specifically described herein. It is therefore intended that the protection granted by letters patent hereon be limited only by the appended claims and equivalents thereof.

### REFERENCES

All references cited herein are hereby expressly incorporated by reference.
ABI Prism.RTM. 7700 Sequence Detection User's Manual: Section D, Theory of Operation.'pgs D-1 to D-22. January 1998.
Allen, P.; Worland, S.; Gold, L. (1995). Isolation of high-affinity RNA ligands to HIV-1 integrase from a random pool. Virology. 209(2):327-336.
Andrake, M.; Guild, N.; Hsu, T.; Gold, L.; Tuerk, C.; Karam, J. (1988). DNA polymerase of bacteriophage T4 is an autogenous translational repressor. Proc. Nat. Acad. Sci. U.S.A. 85:7942-46.
Becker-Andre, M (1991) Meth. Mol. Cell. Biol. 2:189-201.
Binkley, J.; Allen, P.; Brown, D. M.; Green, L.; Turek, C.; Gold, L. (1995). RNA ligands to human nerve growth factor. Nucl. Acid Res. 23(16):3198-3205.
Bless, N. M.; Smith, D.; Charlton, J.; Caermak, B. J.; Schmal, H.; Friedl, H. P.; Ward, Pa. (1997). Protective effects of an aptamer inhibitor of neutrophil elastase in lung inflammation injury. Curr. Biol. 7(11):877-880.
Bock, L. C.; Griffin, L. C.; Latham, J. A.; Vermass, E. H.; Toole, J. J. (1992). Selection of single-stranded DNA molecules that bind and inhibit human thrombin. Nature. 355(6360):564-566.
Boutin et al. (1998). Proc. Natl. Acad. Sci. USA, 88:7744-7748.
Brown, D.; Gold, L. (1995). Template recognition by an RNA-dependent RNA polymerase: identification and characterization of two RNA binding sites on Q beta replicase. Biochemistry. 34(45):14765-14774.
Brown, L. F.; Betmar, M.; Claffey, K.; Nagy, J. A.; Feng, D.; Dvorak, A. M.; Dvorak, H. F. (1997.) Vascular permeability factor/vascular endothelial growth factor: a multifunctional angiogenic cytokine. Regulation of Angiogenesis. Edit by Goldberg & E. M. Rosen, Basel, Switzerland. pgs250-252.
Brown, L. F.; Yeo, K. T.; Berse, B.; Yeo, T. K.; Senger D. R.; Dvorak, H. F.; van de Water, L. (1992). Expression of vascular permeability factor (vascular endothelial growth factor) by epidermal keratinocytes during wound healing. J. of Exp. Med. 176(5):1375-1379.
Bruno, J. G. (1997). In-vitro selection of DNA to chloroaromatics using magnetic microbead-based affinity separation and fluorescence detection. Bioch. and Biophy. Res. Comm. 234(1):117-120.
Burke D. H.; Gold L. (1997). RNA aptamers to the adenosine moiety of S-methionine: adenosyl structural inferences from variations on a theme and the reproducibility of SELEX. Nuc. Acids Res. 25(10):2020-2024.
Burgess, G. W., ed. (1988). ELISA Technology in Diagnosis and Research. Graduate School of Tropical Veterinary Science, James Cook University of North Queensland, Townsville, Australia.
Campbell, N. A. (1987). Biology. Chp. 38:811-812. Circulation and Gas Exchange. The Benjamin/Cummings Publishing Company, Inc., Menlo Park, Calif.
Case, M. et al. (1997) Biochem. Soc. Trans. 25:374S.
Chang, T. C.; Huang, S. H. (1997). A modified iummno-polymerase chain reaction for the detection of .beta.-glucuronidase from Escherichia coli. J. of Imm. Meth. 208:35-42.
Charlton, J.; Sennello, J.; Smith, D. (1997). In vivo imaging of inflammation using an aptamer inhibitor of human neutrophil elastase. Chem. & Biol. 4(11):809-816.
Chen, H.; Gold, L. (1994). Selection of high-affinity RNA ligands to reverse transcriptase: inhibition of cDNA synthesis and Rnase H activity. Biochemistry. 33(29):8746-8756.
Chen, H.; McBroom, D. G.; Zhu, Y.; North T. W.; Gold, L. G. (1996). Inhibitory RNA ligand to reverse transcriptase from feline immunodeficiency virus. Biochemistry. 35(21):6923-30.
Connell, G. J.; Yarus, M. (1994). RNAs with dual specificity and dual NAs with similar specificity. Science. 264(5162): 1137-1141.
Connolly, D. T.; Heuvelman, D. M.; Nelson, R.; Olander, J. V.; Eppley, B. L.; Delfino, J. J.; Siegel, N. R.; Leimgruber, R. M.; Feder, J. (1989). Tumor vascular permeability factor stimulates endothelial cell growth and angiogenesis. J. of Clin. Inv. 84(5):1470-1478.
Conrad, R.; Keranen, L. M.; Ellington, A. D.; Newton, A. C. (1994). Isozyme-inhibition specific inhibition of protein kinase C by RNA aptamers. J. of Biol. Chem. 269(51):32051-32054.
Conrad, R. C.; Giver, L.; Tian, Y.; Ellington, A. D. (1996). In Vitro Selection of Nucleic Acid Aptamers That Bind Proteins. Meth. in Enzym. 267:336-367.
D'Andrea et al. (1989). Cell, 57:277-285.
Dang, C.; Jayasena, S. D. (1996). DNA inhibitors of Taq DNA polymerase facilitate detection of low copy number targets by PCR. J. Mol. Biol. 264:268-278.
Daugherty P S, Olsen M J, Iverson B L, Georgiou G. Development of an optimized expression system for the screening of antibody libraries displayed on the Escherichia coli surface. Protein Eng 1999; 12: 613-621.
Davis, K. A.; Bamaby, A.; Lin, Y.; Jayasena, S. D. (1996). Use of a high affinity DNA ligand in flow cytometry. Nuc. Acids Res. 24(4):702-706.
Davis et al. (1991). Science, 253:59-63.
Dertmar, M.; Brown, L. E.; Claffey, K. P.; Yeo, K. T.; Kocher, O.; Jackman, R. W.; Berse, B.; Dvorak, H. E. (1994). Overexpression of vacular permeability factor/vascular endothelial growth factor and its receptors in psoriasis. J of Exp. Med. 180(3): 1141-1146.
DeVries, C.; Escobedo, J. A.; Ueno, H.; Houck, K.; Ferrara, N.; Williams, L. T. (1992). The fins-like tyrosine kinase, a receptor for vascular endothelial growth factor. Science. 255:989-991.
Doudna, J. A.; Cech, T. R.; Sullenger, B. A. (1995). Selection of an RNA molecule that mimcs a major autoantigenic epitope of human insulin receptor. Proc. Nat. Acad. Sci. U.S.A. 92(6):2355-2359.
Drolet, D. W. (1996). Nature Biotechnology, 14:1021-1025.
Drolet, D. W.; Moon-McDermott, L.; Romig, T. S. (1996). An enzyme-linked onligonucleotideassay. Nat.Biot. 14:1021-1025.
Edery et al. (1989). Proc. Natl. Acad. Sci. USA, 86:2112-2116.
Ekins R, Chu F, Biggart E (1990): Multispot, multianalyte, immunoassay. Ann Biol Clin (Paris): 48:655-666.
Ellingtion, A. D. (1994). RNA selection. Aptamers achieve the desired recognition. Curr. Biol. 4(5):427-429.
Ellington, A. D. Aptamers: A workshop in in vitro selection. Center for Aptamer Research, The Institute for Molecular and Cellular Biology, Indiana University.
Ellington, A. D. and Szostak, J. W. (1992). Nature (England), 355:850-852.
Ellington, A. D. and Szostak, J. W. (1990). Nature (England), 346:818-822.
Ellington, A. D.; Szostak, J. W. (1990). In vitro selection of RNA molecules that bind specific ligands. Nature. 346:818-822.
Fancy D A, Denison C, Kim K, Xie Y, Holdeman T, Amini F, Kodadek T (2000). Chem. Biol 7(9):697-708.
Fasco, M. J.; Treanor, C. P.; Spivack, S.; Figge, H. L.; Kaminsky, L. S. (1995). Quantitative RNA-polymerase chain reaction-DNA analysis by capillary electrophoresis and laser-induced fluorescence. Anal. Bioch. 224:140-147.
Feldhaus M J, Siegel R W, Opresko L K, Coleman J R, Feldhaus J M, Yeung Y A, Cochran J R, Heinzelman P, Colby D, Swers J, Graff C, Wiley H S, Wittrup K D. Flow-cytometric isolation of human antibodies from a nonimmune Saccharomyces cerevisiae surface display library. Nat. Biotechnol. 2003; 21: 163-170.
Ferrara, N.; Henzel, W. J. (1989). Pituitary follicular cells secrete a novel heperin-binding growth factor specific for vascular endothelial cells. Bioch. & Bioph. Res. Comm. 161(2):851-858.
Ferre, F. (1992) PCR Methods Applic. 2:1-9.
Fogarty M, (2002) Future Drug Discovery. Systems Biology and Beyond. 2 (1): Supplement.
Francisco J A, Campbell R, Iverson B L, Georgiou G. Production and fluorescence-activated cell sorting of Escherichia coli expressing a functional antibody fragment on the external surface. Proc Natl Acad Sci USA 1993; 90: 10444-10448.
Fukunaga et al. (1990a). Cell, 61:341-350.
Fukunaga et al. (1990b). Proc. Natl. Acad. Sci. USA, 87:8702-8706.
Gearing et al. (1991). EMBO J., 8:3667-3676; 10:2839-2848.
Gibson, U. E. M.; Heid, C. A.; Williams, P. M. (1996). A novel method for real time quantitative rt-PCR. Gen. Meth. 6:995-1001.
Gibson et al. (1996). Genome Methods, 6:995-1001.
Giver, L.; Bartel, D. P.; Zapp, M. L.; Green, M. R.; Ellington, A. D. (1993). Selection and design of high-affinity RNA ligands for HIV-1 Rev. Gene. 137:19-24.
Goodwin et al. (1990). Cell, 60:941-951.
Gorman et al. (1990). Proc. Natl. Acad. Sci. USA, 87:5459-5463.
Greene, R. et al. (1991). Methods (Orlando), 2:75-86
Griswold, W. (1987) J. Immunoassay 8:145-171
Haab B B, Dunham M J, Brown P O (2001): Protein microarrays for highly parallel detection and quantitation of specific proteins and antibodies in complex solutions. Genome Biol 2:RESEARCH0004.1-4.13.
Harlow, E. R. and Lane, D. W., eds. (1988). Antibodies—A Laboratory Manual. Cold Spring
Harbor Laboratory, Chapter 6.
Hatakeyamaetal. (1989). Science, 244:551-556.
Hayashida et al. (1990). Proc. Natl. Acad. Sci. USA, 244:9655-9659.
Heid, C. A.; Stevens, J.; Livak, K. L.; Williams, P. W. (1996). Real time quantitative PCR. Gen. Meth. 6:986-994.
Hendrickson, E. R.; Truby, T. M. H.; Joerger, R. D.; Majarian, W. R.; Ebersole, R. C. (1995). High sensitivity multianalyte immunoassay using covalent DNA-labeled antibodies and polymerase chain reaction. Nuc. Acids Res. 23(3):522-529.
Hendrickson, E. R. (1995). Nucleic Acids Research, 23:522-529.
Hibi et al. (1990). Cell, 63:1149-1157.
Hicke, B. J.; Watson, S. R.; Koenig, A.; Lynott, C. K.; Bargatze, R. F; Chang, Y.; Ringquist, S.; Moon-McDermott, L.; Jennings, S.; Fitzwater, T.; Han, H.; Varki, N.; Albinana, I.; Willis, M. C.; Varki, A.; Parma, D. (1996). DNA aptamers block L-selectin function in vivo: inhibition of human lymphocyte trafficking in SCID mice. J. of Clin. Invest. 98(12):2688-2692.
Hnatowich, D. J.; Mardirossian, G.; Rusckowski, M.; Winnard Jr., P. (1996). Protein labelling via deoxyribonucleic acid hybridization. Nuc. Med. Comm. 17:66-75.
Hoack, K. A.; Leung, D. W.; Rowland, A. M.; Winer, J.; Ferrara, N. (1992). Dual regulation of vascular endothelial growth factor bioavailability by genetic and proteolytic mechanisms. J. of Biol. Chem. 267(36):26031-26037.
Hollinger et al. (1993). Proc. Natl. Acad. Sci. USA 90:6444-6448.
Ishizaki, J.; Nevins, J. R.; Sullneger, B. A. (1996). Inhibition of cell proliferation by an RNA ligand that selectively blocks E2F function. Nat. Med. 2(12):1386-1389.
Itoh et al. (1990). Science, 247:324-328.
Jaeger, J. A.; Turner, D. H.; Zuker M. (1989a). Improved Predictions of Secondary Structures for RNA. Proc. Natl. Acad. Sci. U.S.A. 86:7706-7710.
Jaeger, J. A.; Turner, D. H.; Zuker M. (1989b). Predicting Optimal and Suboptimal Secondary Structure for RNA. Molecular Evolution: Computer Analysis of Protein and Nucleic Acid Sequences. R. F. Doolittle ed. Meth. in Enzym. 183: 281-306.
Jellinek, D.; Green, L. S.; Bell, C.; Janjic, N. (1994). Inhibition of receptor binding by high-affinity RNA ligands to vascular endothelial growth factor. Biochemistry. 33:10450-10456.
Jellinek, D.; Lynott, C. K.; Rifkin, D. B.; Janjic, N. (1993). High-affinity RNA ligands to basic fibroblast growth factor inhibit receptor binding. Proc. Nat. Acad. Sci. U.S.A. 90(23):11227-11231.
Jenison, R. D.; Gill, S. C.; Pardi, A.; Polisky, B. (1994). High-resolution molecular discrimination by RNA. Science. 263(5152):1425-1429.
Jenkins R E, Pennington S R (2001): Arrays for protein expression profiling: Towards a viable alternative to two-dimensional gel electrophoresis? Electrophoresis 1:13-29.
Joerger, R. D. et al. (1995). Clinical Chemistry, 41:1371-1377.
Jones et al. (1986). Nature, 321:522-525.
Jones et al., (1990). Blood, 76:31-35.
Kawazoe, N.; Ito, Y.; Imanishi, Y. (1997). Bioassay using a labeled oligonucleotide obtained by in vitro selection. Biot. Prog. 13(6):873-876.
Kettman J R, Davies T, Chandler D, Oliver K G, Fulton R J (1998): Classification and properties of 64 multiplexed microsphere sets. Cytometry 33:234-243.
Kohler & Milstein. (1975). Nature, 256:495
Kitamura et al. (1991 a). Cell, 66:1165-1174.
Kitamura et al. (1991b). Proc. Natl. Acad. Sci. USA, 88:5082-5086.
Klagsbrun, M.; D'Amore, P. A. (1991). Regulators of angiogenesis. Ann. Rev. of Physiol. 53: 217-239.
Klagsbrun, M.; D'Amore, P. A. (1996). Vascular endothelial growth factor and its receptors. Cytok. & Grow. Fact. Rev. 7(3):259-270.
Kubik, M. F.; Stephens, A. W.; Schneider, D.; Marlar, R. A.; Tasset, D. (1994). High-affinity RNA ligands to human alpha-thrombin. Nucl. Acids Res. 22(13):2619-2626.
Keyt, B. A.; Berleau, L. T.; Nguyen, H. V.; Chen H.; Heinsohn, H.; Vandlen, R.; Ferrara, N. (1996). The carboxyl-terminal domain (111-165) of vascular endothelial growth factor is critical for its migtogenic potency. J. of Biol. Chem. 271(13):7788-7795.
Larsen et al. (1990). J. Exp. Med., 172:1559-1570.
Kellogg, D. (1990) Anal. Biochem. 189:202-208.
Lentz, S. J.; Varricchio, M.; Smith, S. G.; Ficsor, G. (1997). Detection of low copy number inversions in mouse genomic DNA with unidirectional PCR primers. Envir. & Mol. Mut. 30(3):260-263.
Leroith, D.; Bondy, C. (1996). Growth factors and cytokines in health and disease. The vascular endothelial cell growth factor family and its receptors: molecular and biological proterties. JAI press Inc., Greenwich, Conn., pgs 435-436.
Leung et al. (1987). Nature, 330:537-543.
Lin, Y., Sayasena, S. D. (1997). Inhibition of multiple thermostable DNA polymerases by a heterodimeric aptamer. J. Mol. Biol. 271 (1): 100-111.
Lin, Y.; Nieuwlandt, D.; Magallanez, A.; Feistner, B.; Jayasena, S. D. (1996). High-affinity and specific recognition of human thyroid stimulating hormone (hTSH) by in vitro-selected 2′-amino-modified RNA. Nuc. Acids Res. 24(17):3407-3414.
Liu R, Barrick J E, Szostak J W, Roberts R W. Optimized synthesis of RNA-protein fusions for in vitro protein selection. Methods Enzymol. 2000; 318: 268-293.
Lorsch, J. R.; Szostak, J. W. (1994). In vitro selection of RNA aptamers specific for cyanocobalamin. Biochemistry. 33:973-982.
McElhinny, A. (1997) BioTechniques 23:660-662.
Miemeyer, C. M.; Adler, M.; Blohm, D. (1995). Fluorometric polymerase chain reaction (PCR) enzyme-linked immunosorbent assay for quantification of immuno-PCR products in microplates. Anal. Bioch. 246:140-145.
Mill, J. W. (1997). Vascular endothelial growth factor and ocular neovascularization. Amer. J. of Path. 151(1):13-23.
Miller J C, Zhou H, Kwekel J, Cavallo R, Burke J, Butler E B, Teh B S, Haab B B (2003): Antibody microarray profiling of human prostate cancer sera: Antibody screening and identification of potential biomarkers. Proteomics 3:56-63.
Miller J P, (2002) Analytical & Research Technology. Meeting Today's Analytical Demands of Systems Biology. 3 (1): 16-17.
Miller J P (2002) The Scientist. Bridging Genomics and Proteomics. 16 (15):35.
Morrison et al. (1984). Proc. Natl. Acad. Sci. USA, 81:6851-6855.
Moses, M. A.; Klagsbrun, M.; Shing, Y. (1995). The role of growth factors in vascular cell development and differentiation. Int. Rev. of Cyt. 161:1-48.
Mosley et al. (1989). Cell, 59:335-348.
Mullis, K. B.; Faloona, F. A. (1987). Specific synthesis of DNA in vitro via a polymerase-catalyzed chain reaction. Meth. in Enz. 155:225-250.
Mweene, A. (1996) J. Clin. Microbiol. 334:748-750.
Myoken, Y.; Kayada, Y.; Okamoto, T.; Kan, M.; Sato, G. H.; Sato, J. D. (1991). Vascular endothelial cell growth factor (VEGF) produced by A-431 human epidermoid carcinoma cells and identification of VEGF memebrane binding sites. Proc. Nat. Acad. Sci. U.S.A. 88(13):5819-5823.
Nicewarner-Pena S R, Freeman R G, Reiss B D, He L, Pena D J, Walton I D, Cromer R, Keating C D, Natan M J (2001): Submicrometer metallic barcodes. Science 294:137-141.
Nicola et al (1991). Cell, 67:1-4.
Niemeyer, C. et al. (1997) Anal. Biochem 246:140-145.
Numata, Y.; Matsumoto, Y. (1997). Rapid detection of .varies.-human atrial natriuretic peptide in plasma by a sensitive immuno-PCR sandwich assay. Clin. Chim. Acta 259:169-176.
O'Connor, T., Kane, M. M., and Gosling, J. P. (1995) The dependence of the detection limit of reagent-limited immunoassay on antibody affinity. Biochem. Soc. Trans. 23(2): 393S.
Pang, S. (1990) Nature 343:85-89.
Peterson, E. T.; Blank, J.; Sprinzl, M.; Uhienbeck, O. C. (1993). Selection for active E. coli tRNA(Phe) variants from a randomized library using two proteins. EMBO J. 12(7):2950-2967.
Peterson, E. T.; Pan, T.; Coleman, J.; Uhlenbeck, O. C. (1994). In vitro selection of small RNAs that bind to Escherichia coli phenylalanyl-tRNA synthetase. J. Mol. Biol. 242(3):186-192.
Piatak, M et al. (a) BioTechniques 14:70-81; (b) Science 259:1749-1754.
Pluckthun. (1994). The Pharmacology of Monoclonal Antibodies, vol. 113. Rosenburg and Moore eds. Springer-Verlag, New York, pp. 269-315.
Presta. (1992). Curr. Op. Struct. Biol., 2:593-596.
Quinn, T. P.; Peters, K. G.; DeVries, C.; Ferrara, N.; Williams, L. T. (1993). Fetal liver kinase 1 is a receptor for vascular endothelial growth factor and is selectively expressed in vascular endothelium. Proc. Nat. Acad. Sci. U.S.A. 90(16):7533-7537.
Raeymaekers, L. (1995) Genome Res. 5:91-94.
Reichmann et al. (1988). Nature, 332:323-329.
Renault et al. (1992). Proc. Natl. Acad. Sci. USA, 89:5690-5694.
Ringquist, S.; Jones, T.; Snyder, E. E.; Gibson, T.; Boni, I.; Gold, L. (1995). High-affinity RNA ligands to Escherichia coli ribosomes and ribosomal protein S1: comparison of natural and unnatural binding sites. Biochemistry. 34(11):3640-3648.
Ririe, K., Rasmussen, P. P., and Wittwer, C. T. (1977) Product differentiation by analysis of DNA melting curves during the polymerase chain reaction. Anal. Biochem. 245: 154-160.
Roberts R W and Ja W W (1999). Curr Opin Struct Biol. 9(4):521-9.
Rose et al. (1991). Proc. Natl. Acad. Sci. USA, 88:8641-8645.

Ruzicka, V. et al. (1993) Science 260:698-699.
Saiki, R. K.; Gelfand, D. H.; Stoffel, S.; Scharf, S. J.; Higuchi, R.; Horn, G. T.; Mullis, K. B.; Erlich, H. A. (1988). Primer-directed enzymatic amplification of DNA with a thermostable DNA polymerase. Science. 239:487-491.
Saiki, R. K.; Scharf, S.; Faloona, F.; Mullis, K. B.; Hom, G. T.; Erlich, H. A.; Arnheim, N. (1985). Enzymatic amplification of P-globin genomic sequences and restriciton site analysis for diagnosis of sickle cell anemia. Science. 230:1350-1354.
Sanna, P. P.; Weiss, F.; Samson, M. E.; Bloom, F. E.; Pich, E. M. (1995). Rapid induction of tumor necrosis factor a in the cerebrospinal fluid after intracerebroventricular injection of lipopolysaccharide revealed by a sensitive capture immuno-PCR assay. Proc. Natl. Acad. Sci. U.S.A. 92:272-275.
Sano, T.; Smith, C. L.; Cantor, C. R. (1992). Immuno-PCR: very sensitive antigen detection by means of specific antibody-DNA conjugates. Science. 258:120-122.
Sassanfar, M.; Szostak, J. (1993). An RNA motif that binds ATP. Nature. 364(6437):550-553.
Schaffitzel C, Hanes J, Jermutus L, Pluckthun A. Ribosome display: an in vitro method for selection and evolution of antibodies from libraries. J Immunol Methods 1999; 231: 119-135.
Schaffitzel C, Berger I, Postberg J, Hanes J, Lipps H J, Pluckthun A. In vitro generated antibodies specific for telomeric guanine-quadruplex DNA react with Stylonychia lemnae macronuclei. Proc. Natl. Acad. Sci. U.S. A 2001; 98: 8572-8577.
Schneider, D.; Gold, L.; Platt, T. (1993). Selective enrichment of RNA species for tight binding to Escherichia coli rho factor. FASEB J. 7(1):201-207.
Schneider, D.; Tuerk, C.; Gold, L. (1992). Selection of high affinity RNA ligands to the bacteriophage R17 coat protein. J. Mol. Biol. 228(3):862-869.
Shweiki, D.; Itin, A.; Soffer, D.; Keshet, E. (1992). Vascular endothelial growth factor induced by hypoxia may mediate hypoxia-initiated angiogenesis. Nature. 259:843-845.
Skoda, R. C. et al. (1993). EMBO J. 12:2645-2653.
Smith G P. Filamentous fusion phage: novel expression vectors that display cloned antigens on the virion surface. Science 1985; 228: 1315-1317.
Souyri, M. et al. (1990). Cell 63:1137.
Sperl, J.; Paliwal, V.; Ramabhadran, R.; Nowak, B.; Askenases, P. W. (1995). Soluble T cell receptors: detection and quantitative assay in fluid phase via ELISA or immuno-PCR. J. of Imm. Met. 186:181-194.
Suzuki, A. (1995) Jpn. J. Cancer Res. 86:885-889.
Szostak, J. W. (1992). In vitro genetics. TIBS. 17:89-93.
Takaki et al. (1990). EMBO J., 9:4367-4374.
Takeshita et al. (1991). Science, 257:379-382.
Tavemier et al. (1991). Cell, 66:1175-1184.
Tijssen, P., Practice and Theory of Enzyme Immunoassays. in Laboratory Techniques in Biochemistry and Molecular Biology, vol. 15, ed. by Burdon, R. H. and van Knippenberg, P. H., Elsevier, N.Y., 1985, pp. 132-136.
Tischer, E.; Gospodarowicz, D.; Mitchell. R.; Silva, M.; Schilling, J.; Lau, K.; Crisp, T.; Fiddes, J. C.; Abraham, J. A. (1989). Vascular endothelial growth factor: a new member of the platelet-derived growth factor gene family. Bioch. & Bioph. Res. Comm. 165(3):1198-1206.
Tsai, D. E.; Kenan, D. J.; Deene, J. D. (1992). In vitro selection of an RNA epitope immunologically cross-reactive with a peptide. Proc. Natl. Acad. Sci. U.S.A. 89(19):8864-8868.
Tuerk, C.; Gold, L. (1990). Systematic evolution of ligands by exponential enrichment: RNA ligands to bacteriophage T4 DNA polymerase. Science. 249:505-510.
Turek, C.; MacDougal-Waugh, S. (1993). In vitro evolution of functional nucleic acids: high-affinity RNA ligands of HIV-1 proteins. Gene. 137:33-39.
Tyagi S., and Kramer F R. (1996) Molecular Beacons: Probes that fluoresce upon hybridization. Nature Biotechnology 14(3): 303-308.
Vaisman, N.; Gospodarowicz, D.; Neufled, G. (1990). Characterization of the receptors for vascular endothelial growth factor. J. of Biol. Chem. 265(32):19461-19466.
Vigon, I. et al. (1992). Proc. Natl. Acad. Sci. 89:5640.
Waltenberger, J.; Claesson-Welsh, L.; Siegbahn, A.; Shibuya, M.; Heldin, C. H. (1994). Different signal transduction properties of KDR and flt-i, two rectoptors for vascular endothelial growth factor. J. of Biol. Chem. 269(43):26988-26995.
Wiegand, T. W.; Williams, P. B.; Dreskin, S. C.; Jouvin, M.; Kinet, J.; Tasset, D. (1996). High-affinity oligonucleotide ligands to human IgE inhibit binding to FcE recoptor I. J. of Imm. 157(1):221-230.
Williams, S.; Schwer, C.; Kirshnarao, A.; Heid, C.; Karger, B.; Williams, P. M. (1996). Quantitative competitive PCR: Analysis of amplified products of the HIV-1 gag gene by capillary electrophoresis with laser induced fluorescence detection. Anal. Bioch. 236(1):145-152.
Winter G, Griffiths A D, Hawkins R E, Hoogenboom H R. Making antibodies by phage display technology. Annu. Rev. Immunol. 1994; 12: 433-455.
Wittrup K D. Protein engineering by cell-surface display. Curr. Opin. Biotechnol. 2001; 12: 395-399.
Wittwer, C., Herrmann, M., Moss, A., and Rasmussen, R. (1977) Continuous fluorescence monitoring of rapid cycle DNA amplification. BioTechniques 22(1): 130-138;
   Wittwer, C., Ririe, K., Andrew, R., David, D., Gundry, R., and Balis, U.(1997) The LightCycler™: A microvolume multisample fluorimeter with rapid temperature control. BioTechniques 22(1): 176-181.
Wyatt, J. R.; Pulglisi, J. D., Tinoco, I. (1989). RNA folding: psuedoknots, loops and bulges. BioEssays. 11 (4):100-106.
Xu L, Aha P, Gu K, Kuimelis R, Kurz M, Lam T, Lim A, Liu H, Lohse P, Sun L, Weng S, Wagner R, Lipovsek D. Directed evolution of high-affinity antibody mimics using mRNA display. Chem Biol. 2002; 9: 933-942.
Yamasaki et al. (1988). Science, 241:825-828.
Zapata et al. (1995). Protein Eng. 8(10):1057-1062.
Zhou, H.; Fisher, R. J.; Papas, T. S. (1993). Universal immuno-PCR for ultra-sensitive traget protein detection. Nuc. Acids Res. 21(25):6038-6039.
Zuker, M. (1989). On Finding All Suboptimal Foldings of an RNA Molecule. Science. 244:48-52.

1. A method for quantitating or detecting the presence of a plurality of different target molecules in a sample, comprising:
(a) exposing a sample, which contains or is suspected of containing the target molecules, to one or more different capture molecules capable of binding to the target molecules to form capture molecule:target molecule complexes;
(b) adding to the capture molecule:target molecule complexes a plurality of detector molecules, each detector molecule being capable of specifically binding to a target molecule to form a capture molecule:target molecule:detector molecule ternary complex, wherein each detector molecule comprises a unique nucleic acid sequence tag which is different from the tag on other detector molecule;
(c) separating the detector molecules in said ternary complexes from the unbound detector molecules;
(d) dividing the detector molecules in the ternary complexes of step (c) into a plurality of samples;
(e) performing real time PCR on each of the plurality of samples of step
(d), wherein each PCR reaction has PCR primers specific for one or more nucleic acid sequence tags on the detector molecules; and
(f) analyzing real time PCR data to determine the presence or quantity of the detector molecules and the corresponding target molecules present in the sample.

2. The method of claim 1, further comprising washing the capture molecule:target molecule complexes to remove unbound sample after step (a).

3. The method of claim 1, wherein the step (c) is performed by washing the capture molecule:target molecule:detector molecule complexes to remove the unbound detector molecules.

4. The method of claim 1, wherein the capture molecules are immobilized to a solid support during step (a) or (b).

5. The method of claim 1, wherein the capture molecules are in solution during step (a) or (b), and are immobilized to a solid support after step (b).

6. The method of claim 1, wherein the capture molecules comprise linkers for immobilization to a solid support.

7. The method of claim 1, wherein the capture molecules are labeled with biotin and are bound to a streptavidin or avidin-coated support.

8. The method of claim 1, wherein a plurality of capture molecules are used.

9. The method of claim 1, wherein a single capture molecule is used and wherein the capture molecule is capable of specifically capturing more than one target molecules.

10. The method of claim 1, wherein the capture molecules are antibodies.

11. The method of claim 1, wherein the detector molecules are DNA-labeled antibodies.

12. The method of claim 11, wherein the DNA are linked to the antibodies via linkers.

13. The method of claim 1, wherein the target molecules are proteins or fragments thereof.

14. The method of claim 13, wherein the target molecules are cytokines selected from the group consisting of growth hormone, insulin-like growth factors, human growth hormone, N-methionyl human growth hormone, bovine growth hormone, parathyroid hormone, thyroxine, insulin, proinsulin, relaxin, prorelaxin, glycoprotein hormones, follicle stimulating hormone (FSH), thyroid stimulating hormone (TSH), leutinizing hormone (LH), hematopoietic growth factor, vesicular endothelial growth factor (VEGF), hepatic growth factor, fibroblast growth factor, prolactin, placental lactogen, tumor necrosis factor-alpha, tumor necrosis factor-beta, mullerian-inhibiting substance, mouse gonadotropin-associated peptide, inhibin, activin, vascular endothelial growth factor, integrin, nerve growth factors (NGFs), NGF-beta, platelet-growth factor, transforming growth factors (TGFs), TGF-alpha, TGF-beta, insulin-like growth factor-I, insulin-like growth factor-II, erythropoietin (EPO), osteoinductive factors, interferons, interferon-alpha, interferon-beta, interferon-gamma, colony stimulating factors (CSFs), macrophage-CSF (M-CSF), granulocyte-macrophage-CSF (GM-CSF), granulocyte-CSF (G-CSF), thrombopoietin (TPO), interleukins (ILs), IL-1, IL-1alpha, IL-2, IL-3, IL-4, IL-5, IL-6, IL-7, IL-8, IL-9, IL-11, IL-12, LIF, SCF, neurturin (NTN) and kit-ligand (KL).

15. The method of claim 1, wherein the sample is selected from the group consisting of blood, serum, plasma, sputum, urine, semen, cerebrospinal fluid, sinovial fluid, bronchial aspirate and aqueous extracts from tissues or cells.

16. The method of claim 1, wherein the detection of PCR product in the real time PCR reaction is performed using non-primer probes capable of binding to each nucleic acid tag on the capture molecules, wherein each non-primer probe comprises a nucleic acid having one or more fluorescent dye labels.

17. The method of claim 16, wherein the nucleic acid of each non-primer probe comprises a reporter fluorescent dye and a quencher molecule.

18. The method of claim 1, wherein the nucleic acid tags on the detector molecules are RNA and the RNA nucleic acid tags are reverse transcribed to form DNA before or during amplifying step (e).

19. The method of claim 1, wherein a single nucleic acid sequence tag is analyzed by real time PCR in each real time PCR reaction.

20. The method of claim 1, wherein more than one nucleic acid sequence tag is analyzed by real time PCR in each real time PCR reaction by using different sequence-specific detection probes with different spectrally distinguishable signals.

21. The method of claim 1, wherein the capture molecules bind the target molecules regardless of phosphorylation of the target molecules and each detector molecule specifically binds to a target molecule phosphorylated at a specific site within the target molecule, such that specifically phosphorylated target molecules are quantitated or detected.

22. The method of claim 1, wherein capture molecules specifically bind to target molecules that are phosphorylated at a specific site and the detector molecules bind to the target molecules regardless of the phosphorylation of the target molecules, such that phosphorylated target molecules are quantitated or detected.

23. The method of claim 1, wherein the capture molecules bind to a moiety that is present on a subset of target molecules, and the detector molecules bind to the target molecules regardless of the presence of the moiety.

24. The method of claim 23, wherein the moiety is phosphotyrosine, ubiquitin, a sumo protein, or a form of glycosylation.

25. A method for quantitating or detecting the presence of a plurality of different target molecules in a sample, comprising:
(a) adding to an immobilized sample which contains or is suspected of containing the target molecules a plurality of detector molecules, each detector molecule being capable of specifically binding to a target molecule to form a target molecule:detector molecule complex, wherein each detector molecule comprises a unique nucleic acid sequence tag which is different from the tag on other detector molecule;
(b) washing the surface to remove the non-immobilized detector molecules;
(c) eluting the immobilized detector molecules;
(d) dividing the detector molecules eluted in step (c) into a plurality of samples;
(e) performing real time PCR on each of the plurality of samples in step (d), wherein each PCR reaction has PCR primers specific for one or more nucleic acid sequence tags on the detector molecules; and
(f) analyzing the real time PCR data to determine the presence or amounts of the detector molecules and the corresponding target molecules present in the sample.

26. The method of claim 25, wherein the immobilized sample in step (a) is a cell.

27. The method of claim 26, wherein the target molecules are on the surface of the cell.

28. The method of claim 25, wherein the immobilized sample in step (a) is tissue.

29. The method of claim 25, wherein the sample is immobilized by covalent coupling to the surface.

30. The method of claim 25, wherein the sample is immobilized by non-covalent attachment to the surface.

31. The method of claim 25, wherein a single nucleic acid sequence tag is analyzed by real time PCR in each real time PCR reaction.

32. The method of claim 25, wherein more than one nucleic acid sequence tag is analyzed by real time PCR in each real time PCR reaction by using different sequence-specific detection probes with different spectrally distinguishable signals.

33. A method for quantitating or detecting the presence of a plurality of target molecules on surface of a cell, comprising:
(a) adding to a cell a plurality of detector molecules, each detector molecule being capable of specifically binding to a target molecule to form a target molecule:detector molecule complex, wherein each detector molecule comprises a unique nucleic acid sequence tag which is different from the tag on other detector molecule;
(b) separating the detector molecules that formed complexes with the target molecules on the cell from unbound detector molecules;
(c) dividing the detector molecules that formed complexes with the target molecules on the cell of step (b) into a plurality of samples;
(d) performing real time PCR on each of the plurality of samples of step (c), wherein each PCR reaction has PCR primers specific for one or more nucleic acid sequence tags on the detector molecules; and
(e) analyzing real time PCR data to determine the presence or amounts of the detector molecules and the corresponding target molecules present on the surface of the cell.

34. The method of claim 33, wherein the cells are enriched based on the presence of a cell surface marker before step (a).

35. The method of claim 34, wherein the cells are enriched using a surface coated with a binding molecule that specifically binds to the cell surface marker.

36. The method of claim 35, wherein the surface is a surface from a well of a microtiter plate.

37. The method of claim 35, wherein different cells are enriched using different surface coated with different binding molecules that specifically bind to different cell surface markers.

38. The method of claim 33, wherein a single nucleic acid sequence tag is analyzed by real time PCR in each real time PCR reaction.

39. The method of claim 33, wherein more than one nucleic acid sequence tag is analyzed by real time PCR in each real time PCR reaction by using different sequence-specific detection probes with different spectrally distinguishable signals.


SEQUENCE LISTING (10 sequences)

SEQ ID NO: 1 - DNA, 60 bp, Artificial Sequence
  Synthetic Construct
gcactgctttctactcatcggatgagttgtcatgtcctgcggagaagaaagacggagagt

SEQ ID NO: 2 - DNA, 20 bp, Artificial Sequence
  Synthetic Construct
gcactgctttctactcatcg

SEQ ID NO: 3 - DNA, 20 bp, Artificial Sequence
  Synthetic Construct
actctccgtctttcttctcc

SEQ ID NO: 4 - DNA, 58 bp, Artificial Sequence
  Synthetic Construct
caataaccacccctgaccgatgagttgtcatgtcctgcgcattccttcttctggtcag

SEQ ID NO: 5 - DNA, 18 bp, Artificial Sequence
  Synthetic Construct
caataaccacccctgacc

SEQ ID NO: 6 - DNA, 20 bp, Artificial Sequence
  Synthetic Construct
ctgaccagaagaaggaatgc

SEQ ID NO: 7 - DNA, 60 bp, Artificial Sequence
  Synthetic Construct
cctaccagaccaaggtcaaccgatgagttgtcatgtcctgcggatcattgccctgtgagg

SEQ ID NO: 8 - DNA, 21 bp, Artificial Sequence
  Synthetic Construct
cctaccagaccaaggtcaacc

SEQ ID NO: 9 - DNA, 19 bp, Artificial Sequence
  Synthetic Construct
cctcacagggcaatgatcc

SEQ ID NO: 10 - DNA, 20 bp, Artificial Sequence
  Synthetic Construct
gatgagttgtcatgtcctgc